(19) United States
(12) Patent Application Publication
(10) Pub. No.: US 2024/0374729 A1
(43) Pub. Date: Nov. 14, 2024

(54) COMBINATION OF ANTIGEN SPECIFIC T CELL RECEPTORS AND CHIMERIC CO-STIMULATORY RECEPTORS

(71) Applicant: MEDIGENE IMMUNOTHERAPIES GMBH, Planegg-Martinsried (DE)

(72) Inventors: Maja BÜRDEK, München (DE); Kathrin MUTZE, München (DE); Petra PRINZ, Gilching (DE); Angelika HELMBRECHT, Neufarn (DE)

(21) Appl. No.: 18/686,664

(22) PCT Filed: Aug. 23, 2022
(86) PCT No.: PCT/EP2022/073443

§ 371 Date: Feb. 26, 2024

(30) Foreign Application Priority Data
Aug. 25, 2021 (EP) 21192987.2

(51) Int. Cl. A61K 39/00; A61P 35/00; C07K 14/705; C07K 14/725; C12N 5/0783

(52) U.S. Cl. CPC A61K 39/464489; A61K 39/4611; A61K 39/4632; A61K 39/464488; A61P 35/00; C07K 14/7051; C07K 14/70521; C07K 14/70578; C12N 5/0636; C07K 2319/03

(57) ABSTRACT

The present invention refers immune cells expressing a TCR and a co-stimulatory receptor which are poly functional, i.e. secreting 2 or more proteins. Exemplary immune cells express a(i) T cell receptor (TCR) specific for the PRAME peptide SLLQHLIGL or a TCR specific for NY-ESO-1 peptide SLLMWITQC and (ii) a chimeric co-stimulatory receptor comprising an extracellular domain derived from PD-1 (CD279) and an intracellular domain derived from 4-1BB (CD137).

### FIELD OF THE INVENTION

The present invention refers to immune cells expressing a TCR and a co-stimulatory receptor which are polyfunctional, i.e. secreting 2 or more proteins. Exemplary immune cells express a (i) T cell receptor (TCR) specific for the PRAME peptide SLLQHLIGL or a TCR specific for NY-ESO-1 peptide SLLMWITQC and (ii) a chimeric co-stimulatory receptor comprising an extracellular domain derived from PD-1 (CD279) and an intracellular domain derived from 4-1BB (CD137).

### BACKGROUND OF THE INVENTION

PRAME is a tumor-associated antigen expressed in a wide variety of tumors, preferably melanoma. Further, PRAME has been described as an independent biomarker for metastasis, such as uveal melanoma (Fiedl et al., Clin Cancer Res 2016 March; 22 (5): 1234-1242) and as a prognostic marker for DLBCL (Mitsuhashi et al., Hematology 2014, 1/2014). It is not expressed in normal tissues, except testis. This expression pattern is similar to that of other cancer-testis (CT) antigens, such as MAGE, BAGE and GAGE. The encoded protein acts as a repressor of retinoic acid receptor, and likely confers a growth advantage to cancer cells via this function. Alternative splicing results in multiple transcript variants. PRAME overexpression in triple negative breast cancer has also been found to promote cancer cell motility through induction of the epithelial-to-mesenchymal transition (Al-Khadairi et al., Journal of Translational Medicine 2019; 17:9). Deletion of PRAME has been reported in chronic lymphocytic leukemia, however, this is not functionally relevant since the gene is not expressed in B cells, and the deletion is a consequence of a physiological immunoglobulin light chain rearrangement. Based on the described characteristics of the CT antigen PRAME, it constitutes a suitable target for the treatment of different types of cancers by using TCR-directed cell-based immunotherapies. For this, a TCR with high specificity for the antigen is required that enables the cell product to exert effector functions required for tumor clearance, including release of cytokines, cytotoxicity and proliferation.

NY-ESO-1 and LAGE-1 are important immunotherapeutic target antigens belonging to the family of Cancer/Testis antigens. Cancer/Testis antigens are expressed in various malignant tumors and germ cells of the testis but not on other adult tissues.

Success of immunotherapies with TCR-modified T cells depends not only on the choice of a target antigen but also the selection of a TCR with high antigen specificity and sensitivity. An additional challenge, particularly in treatment of solid tumors, is the immunosuppressive tumor microenvironment (TME) that negatively influences efficacy, fitness and persistence of TCR-modified T cells. In addition to inhibitory cytokines and deprivation of essential metabolic factors, T cells face the inhibitory checkpoint PD-1/PD-L1 axis in the TME that reduces T cell infiltration and causes their exhaustion. Consequently, new strategies are needed to equip TCR-modified T cells with traits to overcome an inhibitory immunosuppressive TME. More specifically, the TCR-modified T cells targeting specific antigens, such as NY-ESO-1 or PRAME with high specificity and with enhanced proliferation, cytokine release and cytotoxicity are desired.

### OBJECTIVES AND SUMMARY OF THE INVENTION

To overcome these needs, the present invention provides a combination of a high avidity TCR and a chimeric co-stimulatory receptor allowing the generation of highly specific T cells targeting antigens, for example PRAME or NY-ESO-1, with enhanced cytokine release, proliferation and cytotoxicity, in particular polyfunctional immune cells secreting 2 or more cytokines.

The invention refers to a target specific immune cell expressing
(A) an antigen specific TCR, and
(B) a chimeric co-stimulatory receptor,
wherein the target specific immune cell secretes at least two proteins.

Thus, one embodiment of the present invention is the provision of a cell comprising
(A) a NY-ESO-1/LAGE-1 specific TCR, comprising
   a TCR α chain comprising a CDR1 having the amino acid sequence of SEQ ID NO: 35, a CDR 2 having the amino acid sequence of SEQ ID NO: 36 and a CDR 3 having the sequence of SEQ ID NO: 37; and
   a TCR β chain comprising a CDR1 having the amino acid sequence of SEQ ID NO: 38, a CDR 2 having the amino acid sequence of SEQ ID NO: 39 and a CDR 3 having the sequence of SEQ ID NO: 40.
      (B) a chimeric co-stimulatory receptor comprising
         an extracellular domain containing a polypeptide derived from PD-1,
         a transmembrane domain, and
         an intracellular domain containing a polypeptide derived from 4-1BB,

The NY-ESO-1-specific TCR used is capable of binding to a NY-ESO-1 peptide having the amino acid sequence SLLMWITQC (SEQ ID NO: 34) or a portion thereof, or its HLA-A2 bound form. It provides high functional avidity and advantageous tumor cell recognition and killing properties. The co-stimulatory receptor reverses the inhibitory checkpoint axis PD-1/PD-L1 to improve the T cell functionality, in particular in a suppressive TME. Thus, the combination of the inventive TCR and the chimeric co-stimulatory receptor allows improved targeting of NY-ESO-1 with high specificity and with enhanced proliferation, cytokine release and cytotoxicity.

Another embodiment of the present invention is the provision of a cell comprising
(A) a PRAME-specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
(B) a chimeric co-stimulatory receptor comprising
   an extracellular domain containing a polypeptide derived from PD-1
   a transmembrane domain, and
   an intracellular domain containing a polypeptide derived from 4-1BB.

The PRAME-specific TCR used is capable of binding to a PRAME peptide having the amino acid sequence SLLQHLIGL (SEQ ID NO: 1) or a portion thereof, or its HLA-A2 bound form. It provides high functional avidity and advantageous tumor cell recognition and killing properties. In particular, the TCR of the present invention has a higher functional avidity than TCRs disclosed in the prior art, recognizes the tested tumor cell lines best, and lyses PRAME positive tumor cells more efficiently. The co-stimulatory receptor reverses the inhibitory checkpoint axis PD-1/PD-L1 to improve the T cell functionality, in particular in a suppressive TME. Thus, the combination of the inventive TCR and the chimeric co-stimulatory receptor allows improved targeting of PRAME with high specificity and with enhanced proliferation, cytokine release and cytotoxicity.

In some embodiments, the PRAME-specific TCR is capable of binding to the HLA-A*02:01, HLA-A*02:02, HLA-A*02:04 or HLA-A*02:09 bound form of SLLQHLIGL. Binding to the PRAME epitope SLLQHLIGL or a portion thereof, or its HLA-A2 bound form induces inter alia IFN-γ secretion by cells transduced or transfected with the TCR.

In some embodiments, the TCR comprises a variable TCR α region having an amino acid sequence which is at least 80% identical to SEQ ID NO: 8 and a variable TCR β region having an amino acid sequence which is at least 80% identical to SEQ ID NO: 9. In more specific embodiments, the TCR comprises a variable TCR α region having the amino acid sequence of SEQ ID NO: 8 and a variable TCR β region having the amino acid sequence of SEQ ID NO: 9. The TCR may comprise a constant TCR α region having an amino acid sequence which is identical or at least 80% identical to SEQ ID NO: 10 and a constant TCR β region having an amino acid sequence which is identical or at least 80% identical to SEQ ID NO: 11.

The chimeric co-stimulatory receptor may comprise a transmembrane domain which is derived from PD-1. In specific embodiments the sequence of chimeric co-stimulatory receptor may comprise the sequence of SEQ ID NO: 26.

Accordingly, a further aspect relates to a composition comprising
a nucleic acid encoding a PRAME-specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Moreover, one aspect relates to a nucleic acid comprising
a nucleic acid encoding a PRAME-specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Accordingly, a further aspect relates to a composition comprising
a nucleic acid encoding a NY-ESO-1specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Moreover, one aspect relates to a nucleic acid comprising
a nucleic acid encoding a NY-ESO-1specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

A further aspect refers to a vector comprising the nucleic acid comprising the sequences for the PRAME-specific TCR and the chimeric co-stimulatory receptor. Another aspect refers to a vector comprising the nucleic acid comprising the sequences for the NY-ESO-1-specific TCR and the chimeric co-stimulatory receptor. Also cells comprising the nucleic acid composition and/or the vector are encompassed.

Typically, the cell is a peripheral blood lymphocyte (PBL) or a peripheral blood mononuclear cell (PBMC). In a specific embodiment, the cell is a T cell.

### Polyfunctional Immune Cells

Thus, one objective of the invention refers to a cell population comprising cells expressing
(A) an antigen specific TCR
(B) a chimeric co-stimulatory receptor
wherein the cell population comprises cells which secrete at least two proteins.

In some embodiments, the chimeric co-stimulatory receptor comprises
an extracellular domain containing a polypeptide derived from PD-1,
a transmembrane domain derived from PD-1, and
an intracellular domain containing a polypeptide derived from 4-1BB, wherein the cell population comprises cells which secrete at least two proteins.

A further aspect of the invention refers to a target specific immune cell expressing
(A) an antigen specific TCR, and
(B) a chimeric co-stimulatory receptor as described herein,
wherein the target specific immune cell secretes at least two proteins.

In some embodiments the cells secrete at least three proteins, such as at least four or at least 5 proteins.

Further aspects refer to a pharmaceutical composition comprising the cell, the composition, the nucleic acid and the vector defined herein. Further aspects refer to the cell, the composition, the nucleic acid and the vector defined herein for cancer treatment.

Surprisingly the inventors found that TCR-T cells expressing chimeric-costimulatory receptor as defined herein show a higher polyfunctionality compared to TCR-T cells lacking chimeric-costimulatory receptor as defined herein. Higher polyfunctionality of transgenic T cells point to a higher functionality and anti-tumor activity in vivo and correlates with clinical outcome.

In particular we could show that a cell population expressing both the target specific, e.g. PRAME specific or NY-ESO-2 specific TCR and the chimeric-costimulatory receptor as defined herein comprises cells which secrete at least two proteins, e.g. at least three proteins, at least four proteins, at least 5 proteins.

### FIGURE LEGENDS

FIG. 1: Co-expression of PD1-41BB does not change TCR expression levels.

CD8+ T cells were isolated from healthy donors and activated with CD3/CD28 antibodies in the presence of IL-7 and IL-15. The activated cells were transduced with retroviral particles containing the sequences for T23.8-2.1-027-004 (=TCR) or a combination of T23.8-2.1-027-004 and PD1-41BB (=TCR_PD1-41BB). Untransduced (=UT) CD8+ T cells that were prepared in the same manner were used as controls. Transduction efficiency and expression levels of the transgenes were determined by antibody staining of the TCR-β chain (TRBV09) and PD-1 and subsequent analysis by flow cytometry.

FIG. 2: Functional avidity of TCR-transgenic T cells is not altered by co-expression of PD1-41BB.

Functional avidities of TCR-transgenic T cell populations are measured as IFN-γ release in co-culture with PD-L1-transgenic T2 cells loaded with titrated amounts of SLLQHLIGL (SLL)-peptide (10-5 M to 10-10 M). The half maximal IFN-γ release serves as measure for functional avidity of the TCR-transgenic effector T cells. Left graph shows absolute values of IFN-γ concentrations determined by ELISA 20 h after co-culture and right graph shows the non-linear regression curve of relative values. While co-expression of PD1-41BB increases IFN-γ levels released by TCR-transgenic T cells in response to PD-L1-positive target cells, the co-expression does not alter the functional avidity of the T cells.

FIGS. 3: HLA-A*02 sub-type recognition is not altered by co-expression of PD1-41BB.

In vitro co-culture of TCR-transduced T cells with selected HLA-A*02 sub-allele-positive lymphoblastoid cell lines (LCL; EBV-transformed B cells) at an E: T ratio of 1:1 (20.000 T cells/well). IFN-γ concentrations were determined by ELISA 20 h after co-culture with LCLs pulsed with 10-5 M SLL-peptide. TCR-transgenic T cells transduced with and without PD1-41BB recognized the SLL-peptide presented by MHC molecules encoded by the HLA-A*02 sub-alleles A*02:02, A*02:04 and A*02:09 at similar levels compared to A*02:01.

FIG. 4: Successful de-risking of potential peptide off-target toxicity.

To decrease the risk for potential off-target toxicities, 191 partially homologous peptides (mismatched (MM) peptides) with up to four amino acid differences compared to the SLL-peptide were selected using Expitope 2.0®. In a pre-screening co-culture using PD-L1-transgenic T2 cells loaded with 10⁻⁶ M of the MM peptides or the SLL-peptide, 33 MM peptides were identified that were recognized by TCR-transduced T cells (data not shown). These 33 MM peptides were examined for their potential to induce IFN-γ release by TCR-transgenic effector T cells when the epitopes (peptides) are endogenously processed by proteasomes of the PRAME-negative target cell line SNB-19. In vitro transcribed (ivt) RNA coding for up to 5 MM peptides was electroporated into SNB-19 cells. The MM peptides that induced the highest IFN-γ release in TCR-transgenic T cells in the pre-screening co-culture (MM01, MM26, MM66), were tested individually as “midigene” constructs (˜400 bp). All other MM peptides were tested as minigene constructs (˜90 bp per peptide) coding for 5 MM peptides. A midigene construct coding for the SLL peptide was used as a positive control. All RNA constructs included an epitope recognized by a positive-control TCR. IFN-γ concentrations were determined 20 h after co-culture of the transfected SNB-19 cells with TCR-transgenic effector T cells. Detected IFN-γ levels indicate no recognition of intracellular processed MM peptides. Therefore, all MM peptides could be de-risked and are not likely to cause potential off-target toxicities. In addition, co-expression of PD1-41BB did not alter the pattern of recognized MM peptides seen with the TCR alone.

FIG. 5: No off-target toxicity was identified using a LCL library covering frequent HLAs.

To assess potential off-target toxicity, TCR-transduced T cells with and without PD1-41BB were co-cultured with a library consisting of 36 lymphoblastoid cell lines (LCL) covering the most frequent HLA-A, -B and -C alleles in the Caucasian population. These LCL express a wide variety of endogenously expressed peptides and help to identify potential cross-reactivities due to recognition of endogenous peptides presented on the matched HLA-A2 molecule or the most frequent other HLA molecules. IFN-γ concentrations were determined by ELISA 20 h after co-culture with LCL. SLL-peptide-loaded HLA-A*02:01-positive LCL served as positive control.

TCR-transgenic T cells secreted very low levels of IFN-γ only when co-cultured with LCL #5, in which low levels of PRAME expression could be confirmed by qPCR. All other LCL were not recognized by the effector T cells expressing the transgenic TCR or the transgenic TCR in combination with PD1-41BB. Therefore, no off-target toxicities could be identified in this safety model.

FIG. 6: No off-target toxicity was identified using a panel of normal cells.

Potential off-target recognition of critical healthy tissues was analyzed by co-culture with normal cells of various tissue origin. As positive controls, normal cells were loaded with SLL-peptide. IFN-γ concentrations in co-culture supernatants were determined by ELISA 20 h after co-culture. No off-target recognition of healthy cells was observed. Only PRAME-positive mature dendritic cells (DC) triggered IFN-γ release in TCR-transgenic T cells above the background of untransduced T cells, whereas the progenitors of mature DC (monocytes and immature DC) did not lead to IFN-γ release by T cells. The addition of PD1-41BB did not change the safety profile of T cells expressing the PRAME-specific TCR. (Human Renal Epithelial Cells (HREpC), Human Renal Cortical Epithelial Cells (HRCEpC), Renal Proximal Tubule Epithelial Cells (RPTEC), Normal Human Lung Fibroblasts (NHLF), Human Osteoblasts (HOB), Monocytes (Mono), immature DC (iDC), mature DC (mDC), iCell Cardiomyocytes2 (iCardio)).

FIG. 7: PD1-41BB enhances the specific release of IFN-γ in response to tumor cells expressing PD-L1.

(A) PRAME-mRNA expression levels in tumor cell lines were determined by real-time quantitative PCR and normalized to the housekeeping gene GUSB. (B) TCR-transgenic T cells with and without PD1-41BB were co-cultured with HLA-A*02:01-positive tumor cell lines of various indications expressing different levels of PRAME and PD-L1. To allow a stable expression of PD-L1 some tumor cells were transduced (TD) with PD-L1. Additionally, it was determined by antibody staining and subsequent flow cytometry analysis that some cell lines showed inducible (ind) PD-L1 expression upon treatment with IFN-γ, while other cell lines already showed some level of endogenous PD-L1 expression (end) without IFN-γ treatment. Untransduced T cells were used as control. IFN-γ concentrations were determined by ELISA 20 h after co-culture. Co-expression of PD1-41BB enhanced the release of IFN-γ in response to PD-L1-positive tumor cells.

FIG. 8: PD1-41BB enhances the specific cytotoxic response against 3-dimensional (3D) tumor cell spheroids.

TCR-transgenic T cells with and without PD1-41BB were co-cultured with 3-dimensional (3D) tumor cell spheroids derived from HLA-A*02:01-positive tumor cell lines expressing different levels of PRAME and PD-L1. Cytotoxicity against the tumor spheroids was determined by loss of red fluorescence over 20 days using Incucyte Zoom® or S3® devices with images being recorded every 4 hours. Fresh tumor cell spheroids were transferred to the co-culture plates on day 3, 7, 10, 13 and 16. Expression of PD1-41BB has a beneficial effect on the effector function and fitness of T cells in a challenging environment with repeated exposure to tumor cells. In the course of multiple challenges with tumor cell spheroids, PD1-41BB-expressing effector T cells can control tumor cell growth better compared to effector T cells expressing only the transgenic TCR.

FIG. 9: PD1-41BB increases the proliferation of TCR-transgenic T cells in response to tumor cells expressing PD-L1.

TCR-transgenic T cells with and without PD1-41BB were co-cultured with HLA-A*02:01-positive tumor cell lines expressing different levels of PRAME and PD-L1 at an effector to target ratio of 1:1. Untransduced T cells were used as control. After 7 days, the X-fold expansion of T cells in the co-culture was calculated from the total cell count. Co-expression of PD1-41BB enhanced the proliferation and/or survival in response to PD-L1-positive tumor cells.

FIG. 10: T cells co-expressing PD1-41BB show strong anti-tumor reactivity in vivo.

5×10⁶ PD-L1-transgenic MelA375 tumor cells were injected subcutaneously into 18 immunodeficient (NOD/Shi-scid/IL-2Rγnull) mice. After one week, mice were distributed to three treatment groups with six mice each. Mice were injected with 10×10⁶ TCR-positive cells (16×10⁶ total cells) with (TCR_PD1-41BB) or without (TCR) PD1-41BB or an equal number of untransduced T cells (UT). Tumor volume was measured 2-3 times a week. PD1-41BB-expressing effector T cells could control tumor cell growth in vivo, whereas effector T cells expressing only the transgenic TCR had little effect compared to untransduced T cells.

FIG. 11: TCR-T cells targeting PRAME expressing PD1-41BB show a higher polyfunctionality compared to TCR-T cells targeting PRAME lacking PD1-41BB. TCR-transgenic T cells with or without PD1-41BB were analyzed regarding their single-cell polyfunctionality (release of 2 or more cytokines) using the IsoLight® technology (IsoPlexis). After co-culture with PD-L1-overexpressing, PRAME-positive MelA375 tumor cells, single CD8+ T cells were evaluated for the secretion of 32 T cell cytokines/proteins and compared with unstimulated TCR-T cells. (A) TCR-T cells expressing PD1-41BB showed a higher percentage of poly-functional T cells compared to TCR-T cells lacking PD1-41BB. Different shades of grey show how many cytokines were released by a single T cell simultaneously. (B) The poly-functional strength index (PSI) was calculated by multiplying the intensities of the various secreted cytokines and the percentage of polyfunctional T cells. TCR-T cells expressing PD1-41BB showed a higher polyfunctional strength index (PSI) compared to TCR-T cells lacking PD1-41BB. Classification of the various cytokines released revealed a high contribution of effector (Gzm-B, IFN-γ, Perforin, TNF-α, TNF-β) and stimulatory (GM-CSF, IL-2, IL-7, IL-8, IL-9, IL-12) cytokines to the superior PSI, followed by chemo-attractive cytokines (IP-10, MIP-1B). Regulatory (IL-10, IL-22) and inflammatory (IL-6, MCP-1) cytokines were released to a lower extent. (C) Detailed analysis of the single-cell poly-cytokine release using a polyfunctionality heat map showed that TCR-T cells with and without PD1-41BB differed in their poly-cytokine signature. Notably, TCR-T cells expressing PD1-41BB contained a higher proportion of single cells secreting 4-10 cytokines simultaneously.

FIG. 12: TCR-T cells targeting NY-ESO-1 expressing PD1-41BB show a higher polyfunctionality compared to TCR-T cells targeting NY-ESO-1 lacking PD1-41BB. TCR-transgenic T cells with or without PD1-41BB were analyzed regarding their single-cell polyfunctionality (release of 2 or more cytokines) using the IsoLight technology (IsoPlexis). After co-culture with PD-L1-overexpressing, NY-ESO-1 positive MelA375 and Mel624.38 tumor cells, single CD8+ T cells were evaluated for the secretion of 32 T cell cytokines/proteins and compared with untransduced TCR-T cells.

(A) TCR-T cells expressing PD1-41BB showed a higher percentage of poly-functional T cells compared to TCR-T cells lacking PD1-41BB. Different shades of grey show how many cytokines were released by a single T cell simultaneously.

(B) The poly-functional strength index (PSI) was calculated by multiplying the intensities of the various secreted cytokines and the percentage of polyfunctional T cells. TCR-T cells expressing PD1-41BB showed a higher polyfunctional strength index (PSI) compared to TCR-T cells lacking PD1-41BB. Classification of the various cytokines/lytic proteins released revealed a high contribution of effector (Gzm-B, IFN-γ, MIP-1a, Perforin, TNF-α, TNF-β) and stimulatory (GM-CSF, IL-2, IL-5, IL-8, IL-9, IL-12) cytokines/lytic proteins to the superior PSI, followed by chemo-attractive (IP-10, MIP-1B, RANTES) cytokines. Regulatory (IL-4, IL-10, IL-22, sCD137, TGF-β1) and inflammatory (IL-6, IL-17F, MCP-1) cytokines were released to a lower extent.

(C) Detailed analysis of the single-cell poly-cytokine release using a polyfunctionality heat map showed that TCR-T cells with and without PD1-41BB differed in their poly-cytokine signature. Notably, TCR-T cells expressing PD1-41BB contained a higher proportion of single cells secreting 2-6 cytokines simultaneously.

### DETAILED DESCRIPTION OF THE INVENTION

Before the invention is described in detail with respect to some of its preferred embodiments, the following general definitions are provided.

The present invention as illustratively described in the following may be suitably practiced in the absence of any element or elements, limitation or limitations, not specifically disclosed herein.

The present invention will be described with respect to particular embodiments and with reference to certain figures, but the invention is not limited thereto but only by the claims.

Where the term “comprising” is used in the present description and claims, it does not exclude other elements. For the purposes of the present invention, the term “consisting of” is considered to be a preferred embodiment of the term “comprising of”′. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense or meaning to the person skilled in the art. If a specific meaning is conveyed to certain terms, definitions of terms will be given in the following in the context of which the terms are used.

### TCR Background

A TCR is composed of two different and separate protein chains, namely the TCR alpha (a) and the TCR beta (β) chain. The TCR α chain comprises variable (V), joining (J) and constant (C) regions. The TCR β chain comprises variable (V), diversity (D), joining (J) and constant (C) regions. The rearranged V (D) J regions of both the TCR α and the TCR β chain contain hypervariable regions (CDR, complementarity determining regions), among which the CDR3 region determines the specific epitope recognition. At the C-terminal region both TCR α chain and TCR β chain contain a hydrophobic transmembrane domain and end in a short cytoplasmic tail.

Typically, the TCR is a heterodimer of one α chain and one β chain. This heterodimer can bind to MHC molecules presenting a peptide.

The term “variable TCR α region” or “TCR α variable chain” or “variable domain” in the context of the invention refers to the variable region of a TCR α chain. The term “variable TCR β region” or “TCR β variable chain” in the context of the invention refers to the variable region of a TCR β chain.

The TCR loci and genes are named using the International Immunogenetics (IMGT) TCR nomenclature (IMGT Database, www.IMGT.org; Giudicelli, V., et al. IMGT/LIGM-DB, the IMGT® comprehensive database of immunoglobulin and T cell receptor nucleotide sequences, Nucl. Acids Res., 34, D781-D784 (2006). PMID: 16381979; T cell Receptor Factsbook, LeFranc and LeFranc, Academic Press ISBN 0-12-441352-8).

### Target

In some embodiments, the TCR provided herein in combination with a chimeric co-stimulatory receptor is advantageously capable of binding to a peptide derived from (human) PRAME (SEQ ID NO: 1) Hence, said TCR is specific for a PRAME peptide as depicted in SEQ ID NO: 1, also called PRAME-SLL. The term “specific for” in the context of the present invention means that the TCR is specifically binding to the target. PRAME (Preferentially Expressed Antigen in Melanoma, Uniprot Acc. No. P78395), also referred to as MAPE (melanoma antigen preferentially expressed in tumors) and OIP4 (OPA-interacting protein 4), has been reported to be a CT antigen with unknown function. PRAME is a Protein Coding gene, associated with Melanoma and Leukemia, and Chronic Myeloid. Gene Ontology (GO) annotations related to this gene include retinoic acid receptor binding. The PRAME protein functions as a transcriptional repressor, inhibiting the signaling of retinoic acid through the retinoic acid receptors RARA, RARB and RARG. It prevents retinoic acid-induced arrest of cell proliferation, differentiation and apoptosis.

In some embodiments, the present invention provides a combination of a chimeric co-stimulatory receptor and a TCR that is capable of binding a peptide comprised within the PRAME amino acid sequence as depicted in SEQ ID NO: 1 (see Table 1). The term “capable of binding” means that said peptide is specifically bound by said TCR. The term “specific (ally) binding” generally indicates that a TCR binds via its antigen binding site more readily to its intended antigenic target than to a random, unrelated non-target antigen. Particularly the term “specifically binds” indicates that the binding specificity of the TCR will be at least about 5-fold, preferably 10-fold, more preferably 25-fold, even more preferably 50-fold, and most preferably 100-fold or more, greater for its antigenic target than its binding specificity for a non-target antigen. The PRAME peptide consisting of the amino acid sequence as depicted in SEQ ID NO: 1 is also referred to as “antigenic target” or “SLL peptide” herein. Hence, the PRAME peptide consisting of the amino acid sequence as depicted in SEQ ID NO: 1 is or comprises the targeted epitope of the TCR of the present invention.

The term “epitope” in general refers to a site on an antigen, typically a (poly-) peptide, which a binding domain recognizes. The term “binding domain” in its broadest sense refers to an “antigen binding site”, i.e. characterizes a domain of a molecule which binds/interacts with a specific epitope on an antigenic target. An antigenic target may comprise a single epitope, but typically comprises at least two epitopes, and can include any number of epitopes depending on the size, conformation, and type of antigen. The term “epitope” in general encompasses linear epitopes and conformational epitopes. Linear epitopes are contiguous epitopes comprised in the amino acid primary sequence and typically include at least 2 amino acids or more. Conformational epitopes are formed by non-contiguous amino acids juxtaposed by folding of the target antigen, and in particular target (poly-) peptide.

The present inventors have found that the minimal amino acid sequence recognized by the TCR of the invention corresponds to the amino acid sequence of PRAME (SEQ ID NO: 1). Specifically, the inventive TCR has been shown to (specifically) recognize the amino acid sequence comprising or consisting of the amino acid sequence SLLQHLIGL (SEQ ID NO: 1), in particular its HLA-A2 bound form as shown in the appended examples. This selective recognition can be obtained by the recognition motif of the TCR, displaying only a few fixed positions. The amino acids LLQ and especially HLI of the sequence SLLQHLIGL (SEQ ID NO: 1) are part of this recognition motif.

Some embodiments refer to a cell comprising
(A) a PRAME-specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
(B) a chimeric co-stimulatory receptor comprising
   an extracellular domain containing a polypeptide derived from PD-1
   a transmembrane domain, and
   an intracellular domain containing a polypeptide derived from 4-1BB, wherein the cell secretes more than two proteins.

Other embodiments refer to a cell comprising
(A) a NY-ESO1-specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40 and
(B) a chimeric co-stimulatory receptor comprising
   an extracellular domain containing a polypeptide derived from PD-1
   a transmembrane domain, and
   an intracellular domain containing a polypeptide derived from 4-1BB, wherein the cell secretes more than two proteins.

### TCR Specific Sequence

Thus an exemplary TCR used in the combination of the invention comprises
a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; or

In some embodiments, the TCR comprises the TCR comprises a variable TCR α region having an amino acid sequence which is at least 80% identical to SEQ ID NO: 8 and a variable TCR β region having an amino acid sequence which is at least 80% identical to SEQ ID NO: 9.

“At least 80% identical”, in particular “having an amino acid sequence which is at least 80% identical” as used herein includes that the amino acid sequence is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set out.

The determination of percent identity between multiple sequences is preferably accomplished using the AlignX application of the Vector NTI Advance™ 10 program (Invitrogen Corporation, Carlsbad CA, USA). This program uses a modified Clustal W algorithm (Thompson et al., 1994.

Nucl Acids Res. 22: pp. 4673-4680; Invitrogen Corporation; Vector NTI Advance™ 10 DNA and protein sequence analysis software. User's Manual, 2004, pp. 389-662). The determination of percent identity is performed with the standard parameters of the AlignX application.

In specific embodiments the TCR comprises a variable TCR α region having the amino acid sequence of SEQ ID NO: 8 and a variable TCR β region having the amino acid sequence of SEQ ID NO: 9.

As can be seen from the examples in some embodiments the TCRs are specific for PRAME, in particular the PRAME epitope SLLQHLIGL (SEQ ID NO: 1) and exhibit only very low cross-reactivity to other epitopes or antigens.

In specific embodiments the TCRs as described herein comprise a constant TCR α region having an amino acid sequence which is at least 80% identical to SEQ ID NO: 10 and a constant TCR β region having an amino acid sequence which is at least 80% identical to SEQ ID NO: 11.

Hence more specifically, in some embodiments the TCR may comprise a variable TCR α region having an amino acid sequence which is at least 80% identical to SEQ ID NO: 8, a variable TCR β region having an amino acid sequence which is at least 80% identical to SEQ ID NO: 9, a constant TCR α region having an amino acid sequence which is at least 80% identical to SEQ ID NO: 10 and a constant TCR β region having an amino acid sequence which is at least 80% identical to SEQ ID NO: 11.

In even more specific embodiments, the TCR may comprise a variable TCR α region having the amino acid sequence of SEQ ID NO: 8, a variable TCR β region having the amino acid sequence of SEQ ID NO: 9, a constant TCR α region having the amino acid sequence of SEQ ID NO: 10 and a constant TCR β region having the amino acid sequence of SEQ ID NO: 11.

Accordingly in specific embodiments, the TCR may comprise a TCR α chain having the amino acid sequence which is identical or which is at least 80% identical to SEQ ID NO: 24, and a TCR β chain having the amino acid sequence which is identical or which is at least 80% identical to SEQ ID NO: 22.

In other embodiments the TCRs are specific for NY-ESO-1, in particular the NY-ESO-1 epitope SLLMWITQC (SEQ ID NO: 34) and exhibit only very low cross-reactivity to other epitopes or antigens.

Thus an exemplary TCR used in the combination of the invention comprises
a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40.

In some embodiments, the TCR comprises a variable TCR α region having an amino acid sequence which is at least 80% identical to SEQ ID NO: 41 and a variable TCR β region having an amino acid sequence which is at least 80% identical to SEQ ID NO: 42.

In specific embodiments the TCR comprises a variable TCR α region having the amino acid sequence of SEQ ID NO: 41 and a variable TCR β region having the amino acid sequence of SEQ ID NO: 42.

In some embodiments, the TCR may comprise a TCR α chain having the amino acid sequence which is identical, or which is at least 80% identical to SEQ ID NO: 43, and a TCR β chain having the amino acid sequence which is identical or which is at least 80% identical to SEQ ID NO: 44.

In some embodiments, the TCR may comprise a TCR α chain having the amino acid sequence of SEQ ID NO: 43, and a TCR β chain having the amino acid sequence of SEQ ID NO: 44.

### Modifications

In some embodiments, the amino acid sequence of the TCR and/or the chimeric co-stimulatory receptor may comprise one or more phenotypically silent substitutions.

“Phenotypically silent substitutions” are also named “conservative amino acid substitutions”. The concept of “conservative amino acid substitutions” is understood by the skilled artisan, and preferably means that codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and codons encoding neutral non-polar residues (A, F, I, L, M, P, V, and W) are substituted with codons encoding neutral non-polar residues. These variations can spontaneously occur, be introduced by random mutagenesis, or can be introduced by directed mutagenesis. Those changes can be made without destroying the essential characteristics of these polypeptides. The ordinarily skilled artisan can readily and routinely screen variant amino acids and/or the nucleic acids encoding them to determine if these variations substantially reduce or destroy the ligand binding capacity by methods known in the art.

The skilled person understands that also the nucleic acid encoding the TCR and/or the chimeric co-stimulatory receptor may be modified. Useful modifications in the overall nucleic acid sequence include codon optimization of the sequence. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region.

According to some embodiments of the invention the amino acid sequence of the TCR and/or the chimeric co-stimulatory receptor is modified to comprise a detectable label, a therapeutic agent or pharmacokinetic modifying moiety.

Non-limiting examples for detectable labels are radiolabels, fluorescent labels, nucleic acid probes, enzymes and contrast reagents. Therapeutic agents which may be associated with the TCRs include radioactive compounds, immune-modulators, enzymes or chemotherapeutic agents. The therapeutic agents could be enclosed by a liposome linked to TCR so that the compound can be released slowly at the target site. This will avoid damage during the transport in the body and ensure that the therapeutic agent, e.g. toxin, has maximum effect after binding of the TCR to the relevant antigen presenting cells. Other examples for therapeutic agents are:

peptide cytotoxins, i.e. proteins or peptides with the ability to kill mammalian cells, such as ricin, diphtheria toxin, pseudomonas bacterial exotoxin A, DNase and RNase. Small molecule cytotoxic agents, i.e. compounds with the ability to kill mammalian cells having a molecular weight of less than 700 Daltons. Such compounds could contain toxic metals capable of having a cytotoxic effect. Furthermore, it is to be understood that these small molecule cytotoxic agents also include pro-drugs, i.e. compounds that decay or are converted under physiological conditions to release cytotoxic agents. Such agents may for example include docetaxel, gemcitabine, cisplatin, maytansine derivatives, rachelmycin, calicheamicin, etoposide, ifosfamide, irinotecan, porfimer sodium photofrin II, temozolomide, topotecan, trimetrexate glucoronate, mitoxantrone, auristatin E, vincristine and doxorubicin; radionuclides, such as, iodine 131, rhenium 186, indium 111, yttrium 90, bismuth 210 and 213, actinium 225 and astatine 213. The association of the radionuclides with the TCRs or derivatives thereof may for example be carried out by chelating agents; immune-stimulators, also known as immunostimulants, i.e. immune effector molecules which stimulate immune response. Exemplary immune-stimulators are cytokines such as IL-2 and IFN-γ, antibodies or fragments thereof, including anti-T cell or NK cell determinant antibodies (e.g anti-CD3, anti-CD28 or anti-CD16); alternative protein scaffolds with antibody like binding characteristics; Superantigens, i.e. antigens that cause non-specific activation of T cells resulting in polyclonal T cell activation and massive cytokine release, and mutants thereof; chemokines such as IL-8, platelet factor 4, melanoma growth stimulatory protein, etc. complement activators; xenogeneic protein domains, allogeneic protein domains, viral/bacterial protein domains, viral/bacterial peptides.

The therapeutic agent may preferably be selected from the group consisting of an immune effector molecule, a cytotoxic agent and a radionuclide. Preferably, the immune effector molecule is a cytokine.

The pharmacokinetic modifying moiety may be for example at least one polyethylene glycol repeating unit, at least one glycol group, at least one sialyl group or a combination thereof. The association of at least one polyethylene glycol repeating unit, at least one glycol group, at least one sialyl group may be caused in a number of ways known to those skilled in the art. In a preferred embodiment the units are covalently linked to the TCR. The TCRs according to the invention can be modified by one or several pharmacokinetic modifying moieties. In particular, the soluble form of the TCR is modified by one or several pharmacokinetic modifying moieties. The pharmacokinetic modifying moiety may achieve beneficial changes to the pharamacokinetic profile of the therapeutic, for example improved plasma half-life, reduced or enhanced immunogenicity, and improved solubility.

The TCR and/or the chimeric co-stimulatory receptor can be modified by attaching additional functional moieties, e.g. for reducing immunogenicity, increasing hydrodynamic size (size in solution) solubility and/or stability (e.g. by enhanced protection to proteolytic degradation) and/or extending serum half-life.

Other useful functional moieties and modifications include “suicide” or “safety switches” that can be used to shut off effector host cells carrying an inventive TCR in a patient's body. An example is the inducible Caspase 9 (iCasp9) “safety switch” described by Gargett and Brown Front Pharmacol. 2014; 5:235. Briefly, effector host cells are modified by well-known methods to express a Caspase 9 domain whose dimerization depends on a small molecule dimerizer drug such as AP1903/CIP, and results in rapid induction of apoptosis in the modified effector cells. The system is for instance described in EP2173869 (A2). Examples for other “suicide” “safety switches” are known in the art, e.g. Herpes Simplex Virus thymidine kinase (HSV-TK), expression of CD20 and subsequent depletion using anti-CD20 antibody or myc tags (Kieback et al, Proc Natl Acad Sci USA. 2008 Jan. 15; 105 (2): 623-8).

TCRs with an altered glycosylation pattern are also envisaged herein. As is known in the art, glycosylation patterns can depend on the amino acid sequence (e.g., the presence or absence of particular glycosylation amino acid residues, discussed below) and/or the host cell or organism in which the protein is produced. Glycosylation of polypeptides is typically either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence.

Another means of glycosylation of TCRs is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. Similarly, deglycosylation (i.e., removal of carbohydrate moieties present on the binding molecule) may be accomplished chemically, e.g. by exposing the TCRs to trifluoromethanesulfonic acid, or enzymatically by employing endo- and exo-glycosidases.

It is also conceivable to add a drug such as a small molecule compound to the TCR, in particular a soluble form of the inventive TCR. Linkage can be achieved via covalent bonds, or non-covalent interactions such as through electrostatic forces. Various linkers, known in the art, can be employed in order to form the drug conjugates.

The TCR, in particular a soluble form of the inventive TCR can additionally be modified to introduce additional domains which aid in identification, tracking, purification and/or isolation of the respective molecule (tags). Thus, in some embodiments, the TCR α chain or the TCR β chain may be modified to comprise an epitope tag.

Epitope tags are useful examples of tags that can be incorporated into the TCR of the invention. Epitope tags are short stretches of amino acids that allow for binding of a specific antibody and therefore enable identification and tracking of the binding and movement of soluble TCRs or host cells within the patient's body or cultivated (host) cells. Detection of the epitope tag, and hence, the tagged TCR, can be achieved using a number of different techniques.

Tags can further be employed for stimulation and expansion of host cells carrying an inventive TCR by cultivating the cells in the presence of binding molecules (antibodies) specific for said tag.

In general, the TCR can be modified in some instances with various mutations that modify the affinity and the off-rate of the TCR with the target antigen. In particular, the mutations may increase the affinity and/or reduce the off-rate. Thus, the TCR may be mutated in at least one CDR and the variable domain framework region thereof.

However, in a preferred embodiment the CDRs of the TCR are not modified or in vitro affinity maturated such as for the TCRs in the examples. This means that the CDRs have naturally occurring sequences. This can be advantageous, since in vitro affinity maturation may lead to immunogenicity to the TCR molecule. This may lead to the production of anti-drug antibodies decreasing or inactivating the therapeutic effect and the treatment and/or induce adverse effects.

The mutation may be one or more substitution(s), deletion(s) or insertions(s). These mutations may be introduced by any suitable method known in the art, such as polymerase chain reaction, restriction enzyme-based cloning, ligation independent cloning procedures, which are described for Example in Sambrook, Molecular Cloning-4th Edition (2012) Cold Spring Harbor Laboratory Press.

Theoretically, unpredictable TCR specificity with the risk for cross-reactivity can occur due to mispairing between endogenous and exogenous TCR chains. To avoid mispairing of TCR sequences, the recombinant TCR sequence may be modified to contain murinized or minimal murinized Ca and CB regions, a technology that has been shown to efficiently enhance correct pairing of several different transduced TCR chains. Murinization of TCRs (i.e. exchanging the human Ca and CB regions by their murine counterparts) is a technique that is commonly applied in order to improve cell surface expression of TCRs in host cells. Without wishing to be bound by specific theory, it is thought that murinized TCRs associate more effectively with CD3 co-receptors; and/or that preferentially pair with each other and are less prone to form mixed TCRs on human T cells genetically modified ex vivo to express the TCRs of desired antigenic specificity, but still retaining and expressing their “original” TCRs.

Nine amino acids responsible for the improved expression of murinized TCRs have been identified (Sommermeyer and Uckert, J Immunol. 2010 Jun. 1; 184 (11): 6223-31) and it is envisaged to substitute one or all of the amino acid residues in the TCRs α and//or β chain constant region for their murine counterpart residues. This technique is also referred to as “minimal murinization”, and offers the advantage of enhancing cell surface expression while, at the same time, reducing the number of “foreign” amino acid residues in the amino acid sequence and, thereby, the risk of immunogenicity.

Some embodiments refer to an isolated TCR as described herein, wherein the TCR is of the single chain type, wherein the TCR α chain and the TCR β chain are linked by a linker sequence.

A suitable single chain TCR form comprises a first segment constituted by an amino acid sequence corresponding to a variable TCR α region, a second segment constituted by an amino acid sequence corresponding to a variable TCR β region fused to the N terminus of an amino acid sequence corresponding to a TCR β chain constant region extracellular sequence, and a linker sequence linking the C terminus of the first segment to the N terminus of the second segment. Alternatively, the first segment may be constituted by an amino acid sequence corresponding to a TCR β chain variable region, the second segment may be constituted by an amino acid sequence corresponding to a TCR α chain variable region sequence fused to the N terminus of an amino acid sequence corresponding to a TCR α chain constant region extracellular sequence. The above single chain TCRs may further comprise a disulfide bond between the first and second chains, and wherein the length of the linker sequence and the position of the disulfide bond being such that the variable domain sequences of the first and second segments are mutually orientated substantially as in native T cell receptors. More specifically the first segment may be constituted by an amino acid sequence corresponding to a TCR α chain variable region sequence fused to the N terminus of an amino acid sequence corresponding to a TCR α chain constant region extracellular sequence, the second segment may be constituted by an amino acid sequence corresponding to a TCR β chain variable region fused to the N terminus of an amino acid sequence corresponding to TCR β chain constant region extracellular sequence, and a disulfide bond may be provided between the first and second chains. The linker sequence may be any sequence which does not impair the function of the TCR.

In the context of the present invention, a “functional” TCR α and/or β chain fusion protein shall mean a TCR or TCR variant, for example modified by addition, deletion or substitution of amino acids, that maintains at least substantial biological activity. In the case of the a and/or β chain of a TCR, this shall mean that both chains remain able to form a TCR (either with a non-modified a and/or β chain or with another inventive fusion protein a and/or β chain) which exerts its biological function, in particular binding to the specific peptide-MHC complex of said TCR, and/or functional signal transduction upon specific peptide: MHC interaction.

In specific embodiments the TCR may be modified, to be a functional TCR α and/or β chain fusion protein, wherein said epitope-tag has a length of between 6 to 15 amino acids, preferably 9 to 11 amino acids. In another embodiment the TCR may be modified to be a functional T-cell receptor (TCR) a and/or β chain fusion protein wherein said TCR α and/or β chain fusion protein comprises two or more epitope-tags, either spaced apart or directly in tandem. Embodiments of the fusion protein can contain 2, 3, 4, 5 or even more epitope-tags, as long as the fusion protein maintains its biological activity/activities (“functional”).

Preferred is a functional TCR α and/or β chain fusion protein according to the present invention, wherein said epitope-tag is selected from, but not limited to, CD20 or Her2/neu tags, or other conventional tags such as a myc-tag, FLAG-tag, T7-tag, HA (hemagglutinin)-tag, His-tag, S-tag, GST-tag, or GFP-tag. myc, T7, GST, GFP tags are epitopes derived from existing molecules. In contrast, FLAG is a synthetic epitope tag designed for high antigenicity (see, e.g., U.S. Pat. Nos. 4,703,004 and 4,851,341). The myc tag can preferably be used because high quality reagents are available to be used for its detection. Epitope tags can of course have one or more additional functions, beyond recognition by an antibody. The sequences of these tags are described in the literature and well known to the person of skill in art.

### Chimeric Co-Stimulatory Receptor

The chimeric co-stimulatory receptor used in combination with the antigen specific TCR (e.g. the PRAME-specific TCR or the NY-ESO specific TCR) comprises
an extracellular domain containing a polypeptide derived from PD-1,
a transmembrane domain, and
an intracellular domain containing a polypeptide derived from 4-1BB.

The chimeric co-stimulatory receptor used in combination with the antigen-specific TCR, such as the PRAME-specific TCR or the NY-ESO-1 specific TCR, herein may particularly comprise an extracellular domain containing the extracellular domain derived from PD-1 (e.g. human PD-1). In this context, the term “derived from” particularly means that the polypeptide contained in the extracellular domain comprises at least a part of PD-1 (e.g. human PD-1), preferably the extracellular domain of PD-1, respectively. The chimeric co-stimulatory receptor comprising an extracellular domain derived from PD-1 has binding activity for PD-L1, PD-L2 or other inhibitory ligands of PD-1. As used herein, the term “derived from” PD-1 also allows that up to 0, 1, 2, 3, 4, 5, 6, 7, 8, 9, 10 or more amino acids are substituted, deleted, and/or inserted compared to a native sequence of PD-1 (e.g. human PD-1) or part thereof (e.g., extracellular domain).

In one embodiment the extracellular domain containing a polypeptide derived from PD-1 comprises sequence set out in SEQ ID NO: 28 or amino acid sequence which is at least 80% identical to SEQ ID NO: 28. In a specific embodiment, the extracellular domain containing a polypeptide derived from PD-1 comprises sequence set out in SEQ ID NO: 28.

In one embodiment of the present invention, the chimeric co-stimulatory receptor comprises an extracellular domain containing a polypeptide derived from PD-1 comprises an amino acid sequence with up to 0, 1, 2, 3, 4, 5, 6, 7, 8, 9, or 10 amino acid substitutions (preferably conservative or highly conservative substitutions), deletions and/or insertions compared to the amino acid sequence of the extracellular domain of human or murine PD-1, e.g., of human PD-1 as depicted in SEQ ID NO: 28.

The chimeric co-stimulatory receptor used in combination with the antigen-specific TCR, such as the PRAME-specific TCR or the NY-ESO-1 specific TCR, herein further comprise a transmembrane domain operably linked between the extracellular domain and the intracellular domain. Generally, the transmembrane domain is not limited to a specific transmembrane domain. Preferably, the transmembrane domain allows stable anchorage of the fusion protein in the membrane of a cell expressing the fusion protein (e.g., a T cell) and further allows binding of the extracellular domain to PD-L1, respectively, and, upon binding to PD-L1, allows signaling transduction to the intracellular domain containing a polypeptide derived from 4-1BB.

In a preferred embodiment, the transmembrane domain of the chimeric co-stimulatory receptor is derived from PD-1. In one embodiment the transmembrane domain comprises a sequence set out in SEQ ID NO: 30 or amino acid sequence, which is at least 80% identical to SEQ ID NO: 30. In a specific embodiment, the transmembrane domain containing a polypeptide derived from PD-1 comprises sequence set out in SEQ ID NO: 30.

The chimeric co-stimulatory receptors used in combination with the antigen-specific TCR, such as the PRAME-specific TCR or the NY-ESO-1 specific TCR, herein may particularly comprise an intracellular domain containing a polypeptide derived from 4-1BB (also termed “41BB”), preferably the intracellular domain of 4-1BB (e.g human 4-1BB). In this context, the term “derived from” particularly means that the polypeptide contained in the intracellular domain comprises at least a part of 4-1BB (e.g. human 4-1BB), preferably the intracellular domain of 4-1BB, respectively. The chimeric co-stimulatory receptor comprising an intracellular domain derived from 4-1BB is capable of increasing the proliferation rate of a T cell expressing said chimeric co-stimulatory receptor upon stimulation with PD-L1, PD-L2 or another inhibitory ligand of PD-1 and/or is capable of increasing the effector function (such as increased IFN-γ release and/or increased cytotoxicity) of a T cell expressing said chimeric co-stimulatory receptor compared to a corresponding T cell not expressing the chimeric co-stimulatory receptor. As used herein, the term “derived from” 4-1BB also allows that up to 0, 1, 2, 3, 4, 5, 6, 7, 8, 9, 10 or more amino acids are substituted, deleted, and/or inserted compared to a native sequence of 4-1BB (human or murine, preferably human 4-1BB) or part thereof (e.g., intracellular domain). In one embodiment the intracellular domain containing a polypeptide derived from 4-1BB comprises sequence set out in SEQ ID NO: 32 or amino acid sequence which is at least 80% identical to SEQ ID NO: 32. In a specific embodiment, the intracellular domain containing a polypeptide derived from 4-1BB comprises sequence set out in SEQ ID NO: 32.

“At least 80% identical”, in particular “having an amino acid sequence which is at least 80% identical” as used herein includes that the amino acid sequence is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set out.

The determination of percent identity between multiple sequences is preferably accomplished using the AlignX application of the Vector NTI Advance™ 10 program (Invitrogen Corporation, Carlsbad CA, USA). This program uses a modified Clustal W algorithm (Thompson et al., 1994. Nucl Acids Res. 22: pp. 4673-4680; Invitrogen Corporation; Vector NTI Advance™ 10 DNA and protein sequence analysis software. User's Manual, 2004, pp. 389-662). The determination of percent identity is performed with the standard parameters of the AlignX application.

### Nucleic Acids, Nucleic Acid Compositions and Vectors

The invention encompasses nucleic acids encoding antigen-specific TCRs, in particular the NY-ESO-1 or PRAME specific TCRs as described herein, as well as the corresponding nucleic acid compositions and vectors comprising said nucleic acids.

The nucleotide sequences encoding the relevant regions and domains of PRAME-specific TCR are set out in Table 1:

TABLE 1

| Peptide seq. | nucleotide seq. | |
|---|---|---|
| SEQ ID NO | SEQ ID NO | Description |
| 2 | 12 | TCR1 α chain CDR1 |
| 3 | 13 | TCR1 α chain CDR2 |
| 4 | 14 | TCR1 α chain CDR3 |
| 5 | 15 | TCR1 β chain CDR1 |
| 6 | 16 | TCR1 β chain CDR2 |
| 7 | 17 | TCR1 β chain CDR3 |
| 8 | 18 | TCR1 α chain variable region |
| 9 | 19 | TCR1 β chain variable region |
| 10 | 20 | TCR α chain constant region |
| 11 | 21 | TCR β chain constant region |
| 22 | 23 | TCR β variable chain |
| 24 | 25 | TCR α variable chain |

The nucleotide sequences encoding the relevant regions and domains of the chimeric co-stimulatory receptor are set out in Table 2:

TABLE 2

| Peptide seq. | nucleotide seq. | |
|---|---|---|
| SEQ ID NO | SEQ ID NO | Description |
| 26 | 27 | chimeric co-stimulatory receptor |
| | | comprising a PD-1 extracellular domain, |
| | | PD-1 transmembrane domain and a 4-1BB |
| | | intracellular domain |
| 28 | 29 | PD-1 extracellular domain |
| 30 | 31 | PD-1 transmembrane domain |
| 32 | 33 | 4-1BB intracellular domain |

Accordingly, a further aspect relates to a composition comprising
a nucleic acid encoding a PRAME-specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Moreover, one aspect relates to a nucleic acid comprising
a nucleic acid encoding a PRAME-specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Moreover, a further aspect relates to a composition comprising
a nucleic acid encoding a NY-ESO-1specific T cell receptor (TCR) comprising
   a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
   a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Moreover, one aspect relates to a nucleic acid comprising
a nucleic acid encoding a NY-ESO-1specific T cell receptor (TCR) comprising
a TCR α chain comprising a CDR1 having the amino acid sequence of SEQ ID NO: 35, a CDR2 having the amino acid sequence of SEQ ID NO: 36 and a CDR3 having the amino acid sequence of SEQ ID NO: 37, and
a TCR β chain comprising a CDR1 having the amino acid sequence of SEQ ID NO: 38, a CDR2 having the amino acid sequence of SEQ ID NO: 39 and a CDR3 having the amino acid sequence of SEQ ID NO: 40; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

A further aspect refers to a vector comprising the nucleic acid comprising the sequences for the PRAME-specific TCR and the chimeric co-stimulatory receptor. Another aspect refers to a vector comprising the nucleic acid comprising the sequences for the NY-ESO-1-specific TCR and the chimeric co-stimulatory receptor. Also cells comprising the nucleic acid composition and/or the vector are encompassed.

“Nucleic acid molecule” generally means a polymer of DNA or RNA, which can be single-stranded or double-stranded, synthesized or obtained (e.g., isolated and/or purified) from natural sources which can contain natural, non-natural or altered nucleotides, and which can contain a natural, non-natural or altered internucleotide linkage, such as a phosphoroamidate linkage or a phosphorothioate linkage instead of the phosphodiester found between the nucleotides of an unmodified oligonucleotide. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g. from Genscript, Thermo Fisher and similar companies). See, for example, Sambrook et al., a nucleic acid can be chemically synthesized using naturally occurring nucleotides or variously modified nucleotides (see for example Sambrook et al. 2001) designed to increase the biological stability of the molecules or to increase the physical stability of the duplex formed upon hybridization (e.g., phosphorothioate derivatives and acridine substituted nucleotides). The nucleic acid can comprise any nucleotide sequence which encodes any of the recombinant TCRs and/or chimeric co-stimulatory receptors, polypeptides, or proteins, or functional portions or functional variants thereof.

For example, the present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding the TCR described herein. Such variant nucleotide sequence encodes a functional TCR that specifically recognizes PRAME or NY-ESO-2, especially PRAME epitope SLLQHLIGL (SEQ ID NO:1) or NY-ESO-1 epitope of SEQ ID NO: 34.

For example, the present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to chimeric co-stimulatory receptor described herein. Such variant nucleotide sequence encodes a functional chimeric co-stimulatory receptor as described herein.

As already described elsewhere herein, the nucleic acid encoding the TCR and/or chimeric co-stimulatory receptor may be modified. Useful modifications in the overall nucleic acid sequence may be codon optimization. Alterations may be made which lead to conservative substitutions within the translated amino acid sequence. With regard to TCRs, these variations can be made in complementarity determining and non-complementarity determining regions of the amino acid sequence of the TCR chain that do not affect function. Usually, additions and deletions should not be performed in the CDR3 region.

Another embodiment refers to a vector comprising the nucleic acid encoding the TCR and the chimeric co-stimulatory receptor as described herein.

The vector is preferably a plasmid, shuttle vector, phagemide, cosmid, expression vector, retroviral vector, adenoviral vector or particle and/or vector to be used in gene therapy.

A “vector” is any molecule or composition that has the ability to carry a nucleic acid sequence into a suitable host cell where synthesis of the encoded polypeptide can take place. Typically, and preferably, a vector is a nucleic acid that has been engineered, using recombinant DNA techniques that are known in the art, to incorporate a desired nucleic acid sequence (e.g. a nucleic acid of the invention). The vector may comprise DNA or RNA and/or comprise liposomes and/viral particles The vector may be a plasmid, shuttle vector, phagemide, cosmid, expression vector, retroviral vector, lentiviral vector, adenoviral vector or particle and/or vector to be used in gene therapy. A vector may include nucleic acid sequences that permit it to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

Preferably, the vector is an expression vector. More preferably, the vector is a retroviral, more specifically a γ-retroviral or lentiviral vector.

The skilled person understands that the chimeric co-stimulatory receptor sequence and the TCR chain TCR-α and TCR-β chain sequences can be included in one nucleic acid, e.g. one vector. In this case the sequences are linked with either an internal ribosomal entry site (IRES) sequence or the 2A peptide sequence derived from a porcine teschovirus (P2A) or derived from other species like Thosea asigna virus 2A peptide (T2A) or foot and mouth disease virus 2A peptide (F2A) (as described in Szymczak et al.: Development of 2A peptide-based strategies in the design of multicistronic vectors) resulting in the expression a single messenger RNA (mRNA) molecule under the control of the viral promoter within the transduced cell.

In specific embodiments, the cell may comprise the nucleic acid encoding the TCR and the chimeric co-stimulatory receptor as described herein or the vector comprising said nucleic acid.

The term “transfection” and “transduction” are interchangeable and refer to the process by which an exogenous nucleic acid sequence is introduced in a host cell, e.g. in a eukaryotic host cell. It is noted that introduction or transfer of nucleic acid sequences is not limited to the mentioned methods but can be achieved by any number of means including electroporation, microinjection, gene gun delivery, lipofection, superfection and the mentioned infection by retroviruses or other suitable viruses for transduction or transfection. The method of cloning and exogenous expression of the TCR is for example described in Engels et al. (Relapse or eradication of cancer is predicted by peptide-major histocompatibility complex affinity. Cancer Cell, 23 (4), 516-26. 2013). The transduction of primary human T cells with a lentiviral vector is, for example, described in Cribbs “simplified production and concentration of lentiviral vectors to achieve high transduction in primary human T cells” BMC Biotechnol. 2013; 13:98.

The cell described and provided in context with the present invention comprising the nucleic acid molecule or the vector as described and provided herein is preferably able to stably or transiently (e.g., stably) express (either constitutively or conditionally) the antigen specific TCR, such as the PRAME-specific TCR or the NY-ESO-1 specific TCR, and the chimeric co-stimulatory receptor of the present invention. The host cell may generally be transduced or transformed by any method with any suitable nucleic acid molecule or vector. In one embodiment, the host cell is transduced with a retroviral or lentiviral (e.g., retroviral) vector comprising a nucleic acid molecule encoding the fusion protein of the present invention or parts thereof (e.g., ECD, TMD, and/or ICD) as described above.

In some embodiments, the cell (including e.g. the target specific immune cell or the cell of the population of cells as referred herein) is a peripheral blood lymphocyte (PBL) or a peripheral blood mononuclear cell (PBMC). The cell may be a natural killer cell or a T cell. Preferably, the cell is a T cell. The T cell may be a CD4+ or a CD8+ T cell. In some embodiments the cell is a stem cell like memory T cell.

Stem cell-like memory T cells (TSCM) are a less-differentiated subpopulation of CD8+ or CD4+ T cells, which are characterized by the capacity of self-renewal and to persist long-term. Once these cells encounter their antigen in vivo, they differentiate further into central memory T cells (TCM), effector memory T cells (TEM) and terminally differentiated effector memory T cells (TEMRA) with some TSCM remaining quiescent (Flynn et al., Clinical & Translational Immunology (2014). These remaining TSCM cells show the capacity to build a durable immunological memory in vivo and therefore are considered an important T cell subpopulation for adoptive T cell therapy (Lugli et al., Nature Protocols 8, 33-42 (2013) Gattinoni et al., Nat. Med. 2011 October; 17 (10): 1290-1297). Immune-magnetic selection can be used in order to restrict the T cell pool to the stem cell memory T cell subtype see (Riddell et al. 2014, Cancer Journal 20 (2): 141-44).

### Pharmaceutical Compositions, Medical Treatments and Kits

Another aspect of the invention refers to pharmaceutical composition comprising the cell comprising the antigen-specific TCR, such as the NY-ESO-1 specific TCR or the PRAME-specific TCR, and the chimeric co-stimulatory receptor or comprising nucleic acid molecules encoding said molecules as described herein, the nucleic acid encoding the antigen-specific TCR, such as the NY-ESO-1 specific TCR or the PRAME-specific TCR, and the chimeric co-stimulatory receptor, a composition comprising nucleic acids encoding the antigen-specific TCR, such as the NY-ESO-1 specific TCR or the PRAME-specific TCR, and nucleic acids encoding the chimeric co-stimulatory receptor, the corresponding vector as described herein.

Those active components of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” defines a non-toxic material, which does not interfere with effectiveness of the biological activity of the active component. The choice of the carrier is dependent on the application.

The pharmaceutical composition may contain additional components which enhance the activity of the active component or which supplement the treatment. Such additional components and/or factors can be part of the pharmaceutical composition to achieve synergistic effects or to minimize adverse or unwanted effects.

Techniques for the formulation or preparation and application/medication of active components of the present invention are published in “Remington's Pharmaceutical Sciences”, Mack Publishing Co., Easton, PA, latest edition. An appropriate application is a parenteral application, for example intramuscular, subcutaneous, intramedular injections as well as intrathecal, direct intraventricular, intravenous, intranodal, intraperitoneal or intratumoral injections. The intravenous injection is the preferred treatment of a patient.

According to a preferred embodiment, the pharmaceutical composition is an infusion or an injection.

An injectable composition is a pharmaceutically acceptable fluid composition comprising at least one active ingredient, e.g. an expanded T cell population (for example autologous or allogenic to the patient to be treated) comprising the antigen-specific TCR, such as the NY-ESO-1 specific TCR or the PRAME-specific TCR, and the chimeric co-stimulatory receptor. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the fusion proteins of this disclosure are conventional; appropriate formulations are well known to those of ordinary skill in the art.

Typically, the pharmaceutical composition comprises at least one pharmaceutically acceptable carrier.

Accordingly, another aspect of the invention refers to the cell as described herein, the composition as described herein, the nucleic acid as described herein, and/or the vector as described herein for use as a medicament.

Some embodiments refer to the to the cell as described herein, the composition as described herein, the nucleic acid as described herein, and/or the vector for use in the treatment of cancer.

In one embodiment the cancer is a hematological cancer or a solid tumor.

Hematological cancers also called blood cancers which do not form solid tumors and therefore are dispersed in the body. Examples of hematological cancers are leukemia, lymphoma or multiple myeloma. There are two major types of solid tumors, sarcomas and carcinomas. Sarcomas are for example tumors of the blood vessel, bone, fat tissue, ligament, lymph vessel, muscle or tendon. In a specific embodiment the cancer is a solid tumor.

In one embodiment, the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, carcinoma, sarcoma or osteosarcoma.

Compositions comprising the modified T cells as described herein can be utilized in methods and compositions for adoptive immunotherapy in accordance with known techniques, or variations thereof that will be apparent to those skilled in the art based on the instant disclosure.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The number of cells for an effective treatment in the composition is typically greater than 10 cells, and up to 10⁶, up to and including 10⁸ or 10⁹ cells and can be more than 10¹⁰ cells. The number of cells will depend upon the ultimate use for which the composition is intended as will the type of cells included therein. For uses provided herein, the cells are generally in a volume of a liter or less, can be 500 ml or less, even 250 ml or 100 ml or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells. Pharmaceutical compositions provided herein can be in various forms, e.g., in solid, liquid, powder, aqueous, or lyophilized form. Examples of suitable pharmaceutical carriers are known in the art. Such carriers and/or additives can be formulated by conventional methods and can be administered to the subject at a suitable dose. Stabilizing agents such as lipids, nuclease inhibitors, polymers, and chelating agents can preserve the compositions from degradation within the body. In a composition intended to be administered by injection, one or more of a surfactant, preservative, wetting agent, dispersing agent, suspending agent, buffer, stabilizer and isotonic agent may be included.

The viral vector particles comprising a nucleotide sequence encoding the antigen-specific TCR, such as the NY-ESO-1 specific TCR or the PRAME-specific TCR, and the chimeric co-stimulatory receptor provided herein, can be packaged as kits. Kits can optionally include one or more components such as instructions for use, devices, and additional reagents, and components, such as tubes, containers and syringes for practice of the methods. Exemplary kits can include the nucleic acids encoding the recombinant TCRs and the chimeric co-stimulatory receptors, the recombinant polypeptides, or viruses provided herein, and can optionally include instructions for use, a device for detecting a virus in a subject, a device for administering the compositions to a subject, and a device for administering the compositions to a subject.

Kits comprising polynucleotides encoding the antigen-specific TCR, such as the NY-ESO-1 specific TCR or the PRAME-specific TCR, and the chimeric co-stimulatory receptor are also contemplated herein. Kits comprising a viral vector encoding a sequence of interest (e.g., a recombinant TCR) and optionally, a polynucleotide sequence encoding an immune checkpoint inhibitor are also contemplated herein.

Kits contemplated herein also include kits for carrying out the methods for detecting the presence of polynucleotides encoding any one or more of the TCRs and/or the chimeric co-stimulatory receptors disclosed herein. In particular, such diagnostic kits may include sets of appropriate amplification and detection primers and other associated reagents for performing deep sequencing to detect the polynucleotides encoding TCRs and/or the chimeric co-stimulatory receptors disclosed herein. In further embodiments, the kits herein may comprise reagents for detecting the TCRs and/or the chimeric co-stimulatory receptors disclosed herein, such as antibodies or other binding molecules. Diagnostic kits may also contain instructions for determining the presence of the polynucleotides encoding the TCRs and/or the chimeric co-stimulatory receptors disclosed herein or for determining the presence of the TCRs and/or the chimeric co-stimulatory receptors disclosed herein. A kit may also contain instructions. Instructions typically include a tangible expression describing the components included in the kit, and methods for administration, including methods for determining the proper state of the subject, the proper dosage amount, and the proper administration method. Instructions can also include guidance for monitoring the subject over the duration of the treatment time.

Kits provided herein also can include a device for administering a composition described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a virus of the kit will be compatible with the virus of the kit; for example, a needle-less injection device such as a high pressure injection device can be included in kits with viruses not damaged by high pressure injection, but is typically not included in kits with viruses damaged by high pressure injection.

### Cytokine Release

The cells expressing both the TCR and the costimulatory molecule, such as PD1-4BB, show enhanced cytokine release. Cytokines/lytic protein that are secreted in high amounts by the population are inter alia IFN-γ, Gzm-B, and IP-10, MIP-1β.

Surprisingly, immune cells, in particular TCR-T cells expressing chimeric-costimulatory receptor as defined herein show a higher polyfunctionality compared to TCR-T cells lacking chimeric-costimulatory receptor as defined herein.

Higher polyfunctionality of transgenic T cells point to a higher functionality and anti-tumor activity in vivo and correlates with clinical outcome.

The term “polyfunctional” means that the cell secretes at least 2, such as at least three, at least four, at least five proteins.

The skilled person understands that the cell secretes the proteins after antigen specific stimulation, for example when the cell is contacted with an antigen presenting cell presenting the antigen in a MHC molecule.

In particular, the cell population comprises a significant percentage of cells, such as 0.1%, 0.2%, 0.3%, 0.4%, 0.5%, 1%, 1.5%, 2%, 3%, 4%, 4.5% polyfunctional cells.

In particular, the cell population comprises a significant percentage of cells expressing at least 1%, preferably at least 1.5%, more preferably 1.75% 3 or more proteins.

In some embodiments the polyfunctional strength index is 80 or more, preferably 90 or more, more preferably 100 or more.

The poly-functional strength index (PSI) is calculated by multiplying the intensities of the various secreted cytokines and the percentage of polyfunctional T cells.

In particular we could show that a cell population expressing both the target specific, e.g. PRAME or NY-ESO-1 specific, TCR and the chimeric-costimulatory receptor as defined herein comprises cells which secrete at least two proteins, e.g. at least three proteins, at least four proteins, at least 5 proteins.

Hence, another aspect of the invention refers to an in vitro assay indicating the potency of a population of T cells by indicating whether a population of T cells comprises a significant proportion of polyfunctional T cells as defined herein.

Thus, in general, desired are highly potent target specific T cells, that secrete at least two, at least three, at least four, at least five proteins selected from the group of effector proteins, stimulatory cytokines and chemo-attractive cytokines.

In particular, the invention refers to an in vitro assay indicating the potency of a population of immune cells, comprising the steps
measuring the proportion of polyfunctional immune cells of a first population of cells,
assessing the potency of the first population of immune cells and assessing the potency of a second population of T cells, wherein an increased proportion of polyfunctional cells in the first population compared the second population indicates that the first population has a higher potency than the second population.

The measurement of the proportion of polyfunctional immune cells may be carried out by the IsoLight® technology (Isoplexis).

Accordingly, T cell populations comprising target specific T cells wherein the cell population comprises T cells that secrete at least two, at least three, at least four, at least five proteins selected from the group of effector proteins, stimulatory cytokines and chemo-attractive cytokines are desired.

The effector proteins may be selected from the group consisting of Gzm-B, IFN-γ, Perforin, TNF-α, TNF-β, MIP-1α.

In one embodiment, the stimulatory cytokines may be selected from the group consisting of GM-CSF, IL-2, IL-7, IL-8, IL-9, IL-12. In one embodiment, the stimulatory protein is selected from GM-CSF, IL-2 and IL-8. In one embodiment the stimulatory protein protein is GM-CSF.

The chemo-attractive cytokines may be selected from IP-10 and MIP-1B.

In some embodiments, each of the cells secreting at least two proteins, secretes at least one of the proteins selected from the group consisting of Gzm-B, IFN-γ, Perforin, TNF-α, TNF-β, MIP-1a, GM-CSF, IL-2, IL-5, IL-7, IL-8, IL-9, IL-12, IP-10 and MIP-1B. In some embodiments, each of the cells secreting at least two proteins, secretes at least one of the proteins selected from the group consisting of Gzm-B, IFN-γ, Perforin, TNF-α, TNF-β, GM-CSF, IL-2, IL-8, MIP-1B and IP-10. In some embodiments, each of the cells secreting at least two proteins, secretes at least one of the proteins selected from the group consisting of IFN-γ, Gzm-B, GM-CSF, MIP-1β and IP-10. In some embodiments, each of the cells secreting at least two proteins, secretes at least one of the proteins selected from the group consisting of IFN-γ, Gzm-B, GM-CSF. In some embodiments, each of the cells secreting at least two proteins, secretes at least one of the proteins selected from the group consisting of IFN-γ, Gzm-B, and IP-10. In some embodiments, each of the cells secreting at least two proteins, secretes at least one of the proteins selected from the group consisting of IFN-γ and Gzm-B.

Typically, the cell population does not comprise cells which secrete significant amounts of IL-6 and/or IL-10.

Thus, some embodiments refer to T cells expressing (A) a PRAME specific TCR as defined herein and (B) a chimeric co-stimulatory receptor as defined herein, wherein the T cells secrete at least two, at least three, at least four, at least five proteins selected from the group consisting of effector proteins, stimulatory cytokines and chemo-attractive cytokines.

Accordingly, some embodiments refer to cell population comprising T cells expressing (A) a PRAME specific TCR as defined herein and (B) a chimeric co-stimulatory receptor as defined herein, wherein the cell population comprises T cells secreting at least two, at least three, at least four, at least five proteins selected from the group consisting of effector proteins, stimulatory cytokines and chemo-attractive cytokines.

Other embodiments refer to T cells expressing (A) a NY-ESO-1 specific TCR as defined herein and (B) a chimeric co-stimulatory receptor as defined herein, wherein the T cells secrete at least two, at least three, at least four, at least five proteins selected from the group consisting of effector proteins, stimulatory cytokines and chemo-attractive cytokines.

Accordingly, some embodiments refer to cell population comprising T cells expressing (A) a NY-ESO-1 specific TCR as defined herein and (B) a chimeric co-stimulatory receptor as defined herein, wherein the cell population comprises T cells secreting at least two, at least three, at least four, at least five proteins selected from the group consisting of effector proteins, stimulatory cytokines and chemo-attractive cytokines

### Experiments

Example 1: Co-Expression of PD1-41BB does not Change TCR Expression Levels.

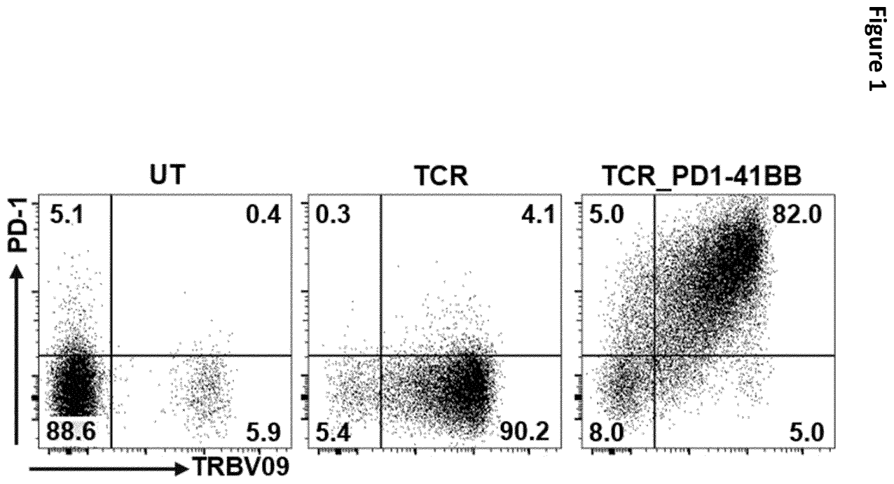

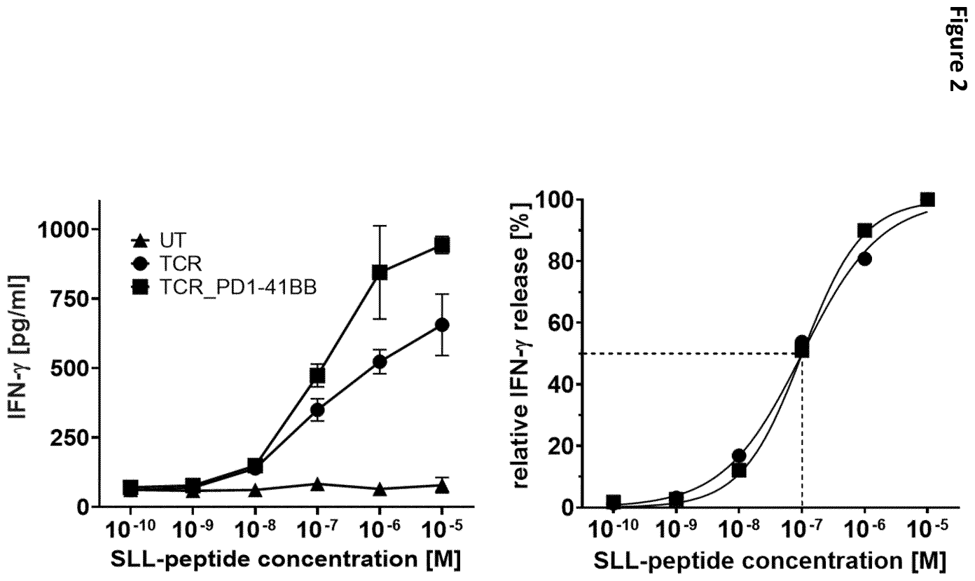

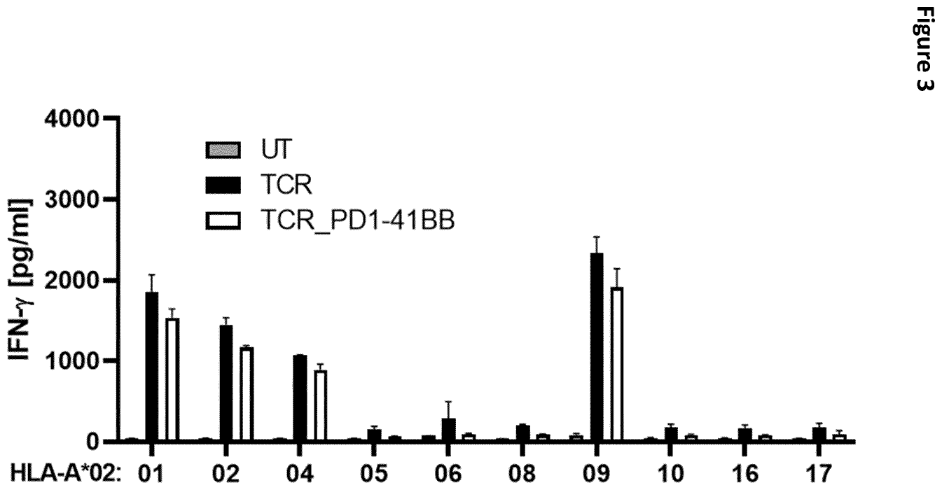

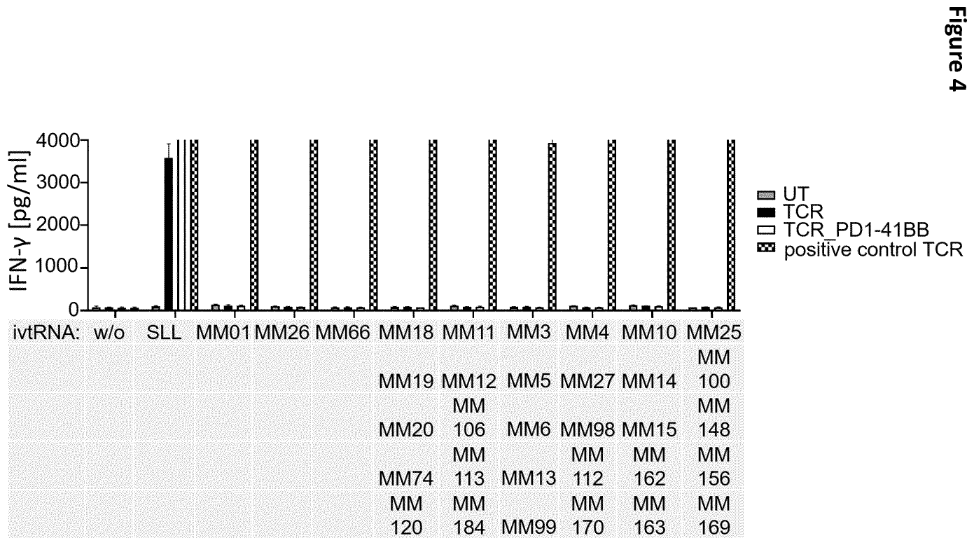

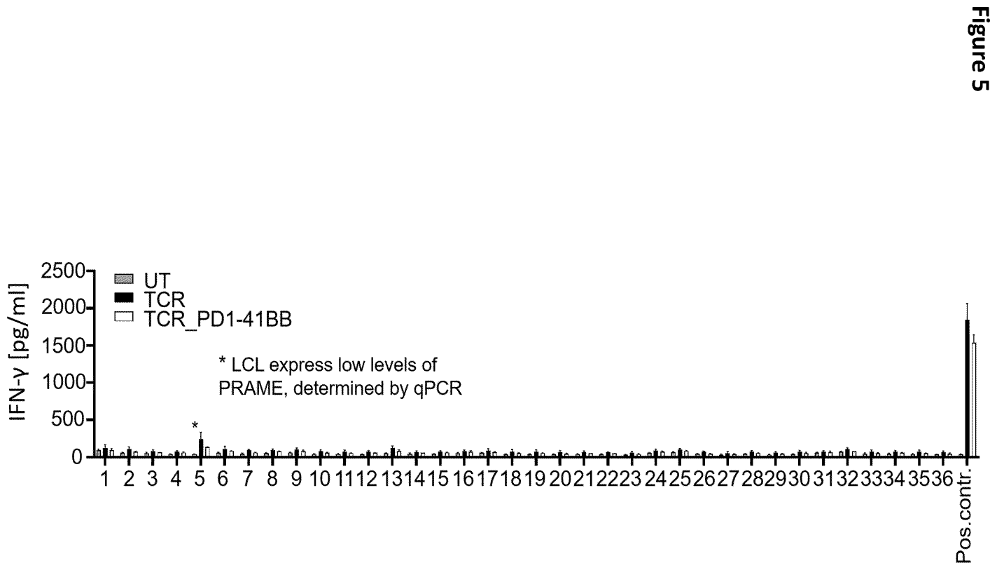

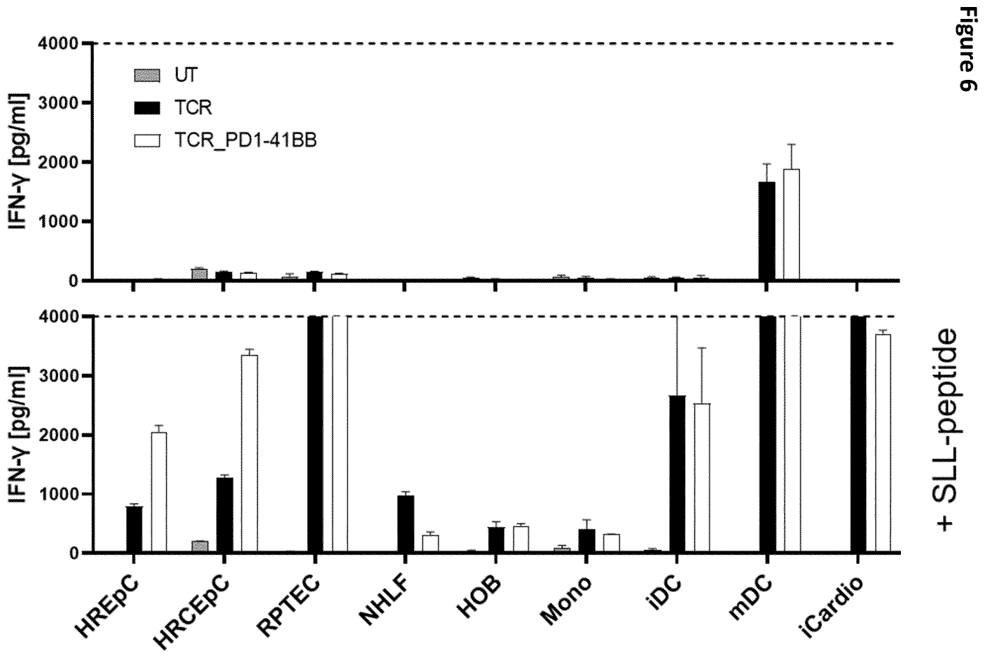

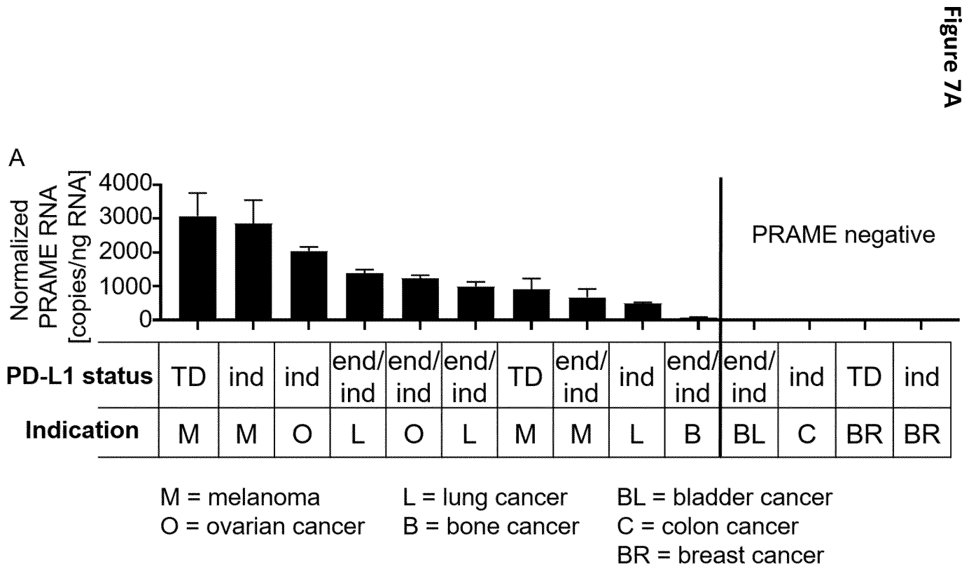

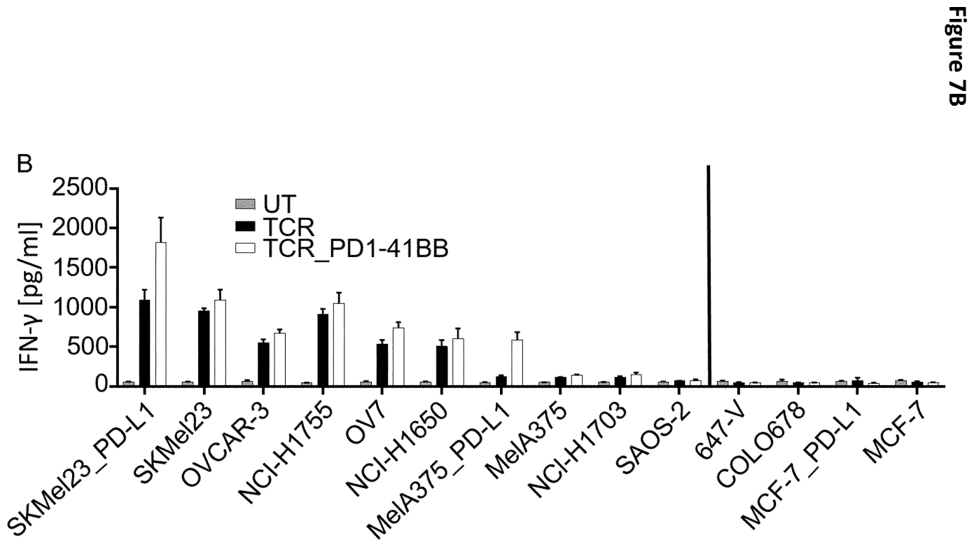

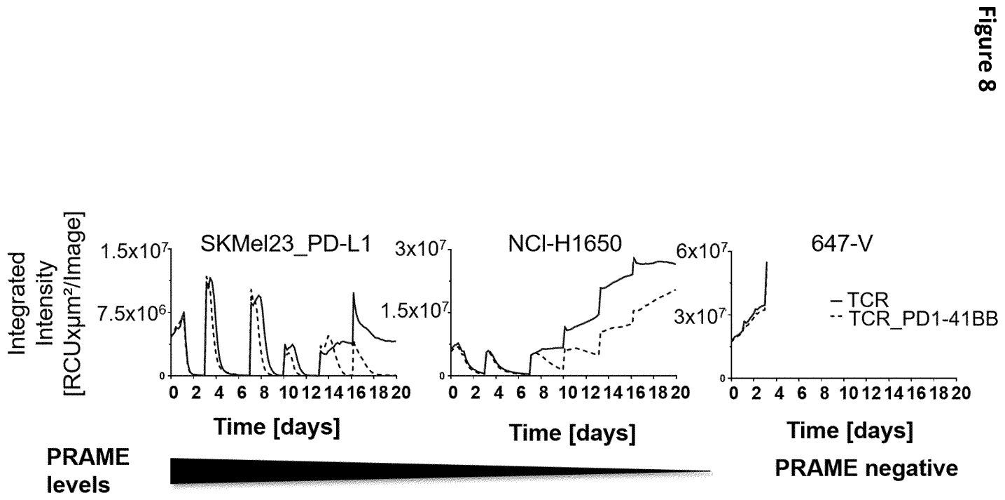

To prepare effector T cells for testing and characterization of the transgenic TCR T23.8-2.1-027-004 (=TCR) and the TCR in combination with PD1-41BB (=TCR_PD1-41BB), CD8+ T cells were isolated from healthy donors and activated with CD3/CD28 antibodies in the presence of IL-7 and IL-15. The activated cells were transduced with retroviral particles containing either the sequence of the TCR or the sequence of the TCR coupled to PD1-41BB. Untransduced (=UT) CD8+ T cells that were prepared in the same manner were used as controls. Transduction efficiency and expression levels of the transgenes was determined on day 14 by antibody staining of the TCR-β chain (TRBV09) and PD-1 and subsequent analysis by flow cytometry. The analysis demonstrates that high transduction rates can be achieved for both constructs TCR (90.2%) and TCR_PD1-41BB (82%) (FIG. 1). PD-1 expression cannot be detected in untransduced (UT) and TCR-transduced effector T cells. However, binding of the PD-1 antibody to TCR_PD1-41BB-transduced T cells indicates high expression levels of PD1-41BB that correlate with the expression of the transgenic TCR. Co-expression of PD-41BB results in TCR expression levels comparable to those measured in effector T cells expressing only the transgenic TCR. This demonstrates that co-expression of the PD1-41BB switch receptor and the transgenic TCR is feasible in T cells and results in equimolar expression on the cell surface.

### Example 2: Functional Avidity of TCR-Transgenic T Cells is not Altered by Co-Expression of PD1-41BB.

Functional avidity refers to the accumulated strength of multiple affinities of individual non-covalent binding interactions, such as the transgenic TCR and the peptide-MHC complex. As such the functional avidity of effector T cells serves as a measure of peptide sensitivity. TCRs conferring a high peptide sensitivity are able to recognize lower amounts of peptide. To investigate whether co-expression of the PD1-41BB receptor influences the peptide sensitivity of TCR-transgenic effector T cells, they were co-cultured with PD-L1-transgenic T2 cells (T2_PD-L1) that carry both the required HLA (HLA-A2: 01) and over-express PD-L1 to allow ligation with PD1-41BB.

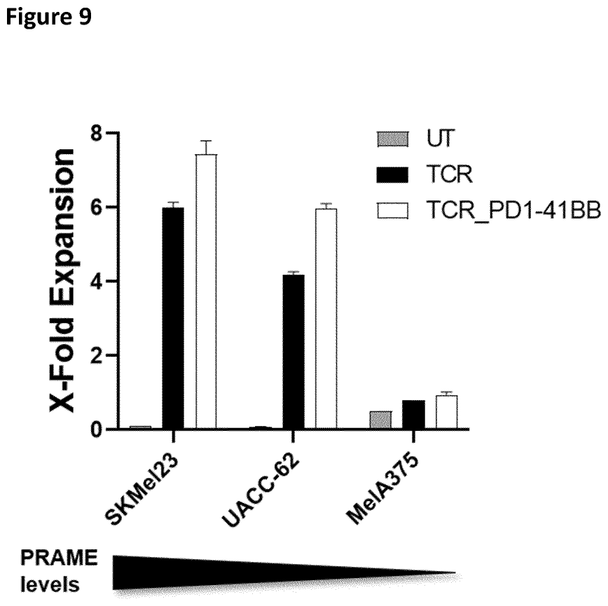

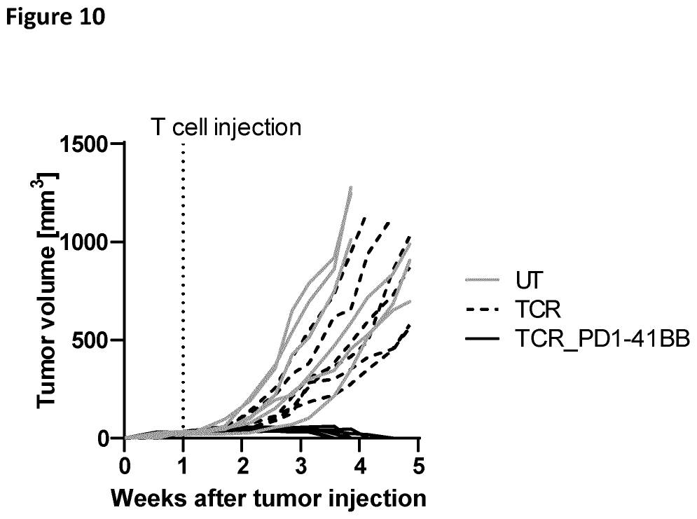

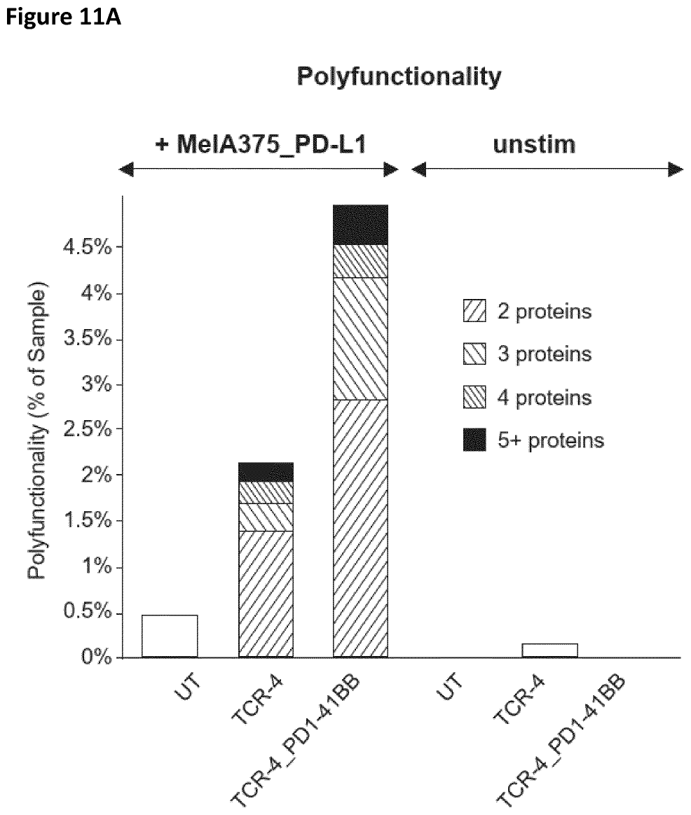

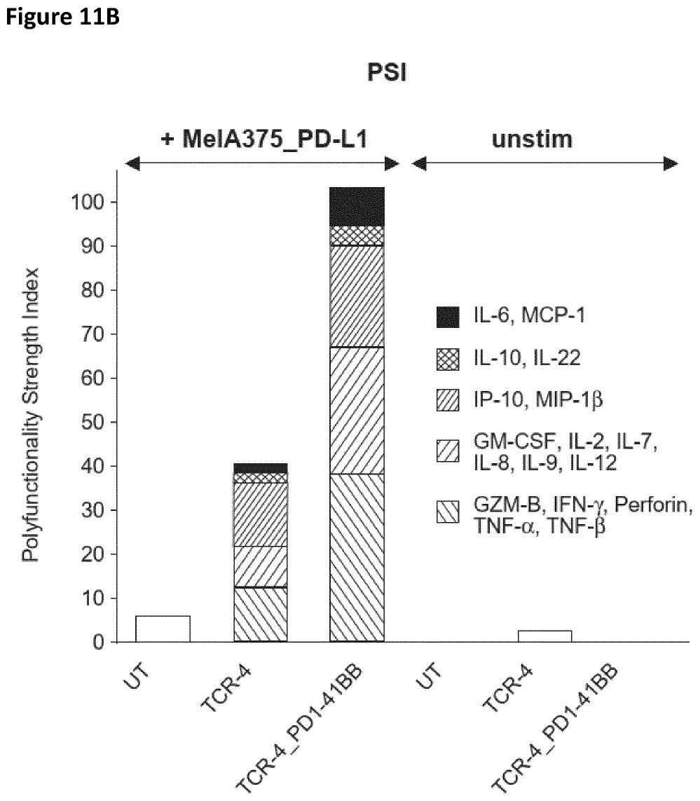

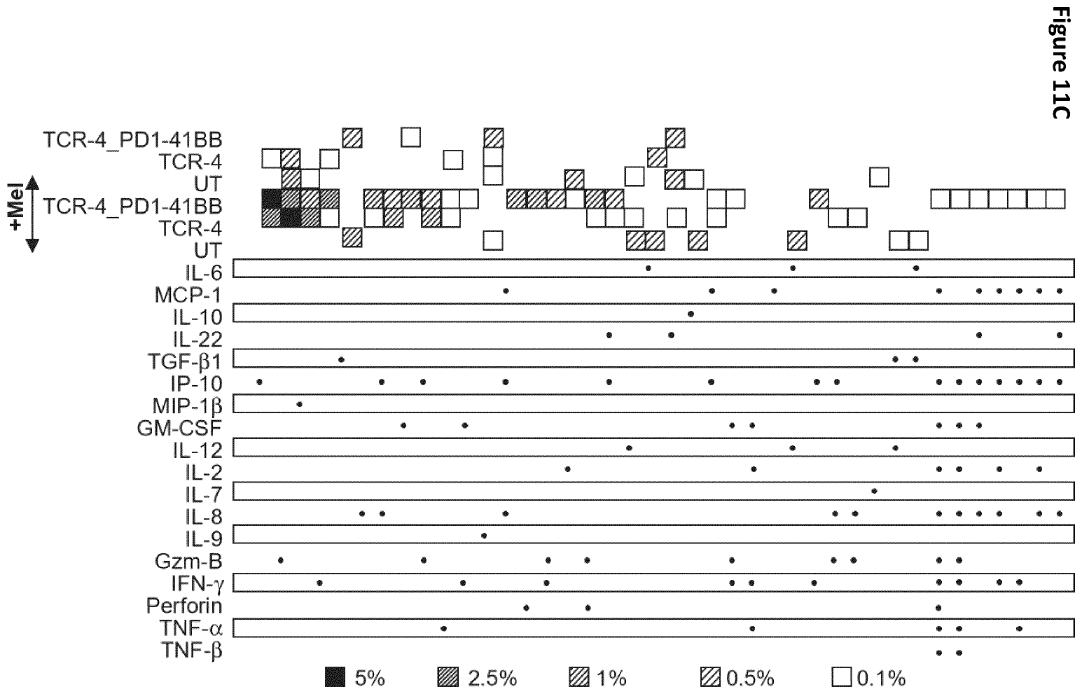

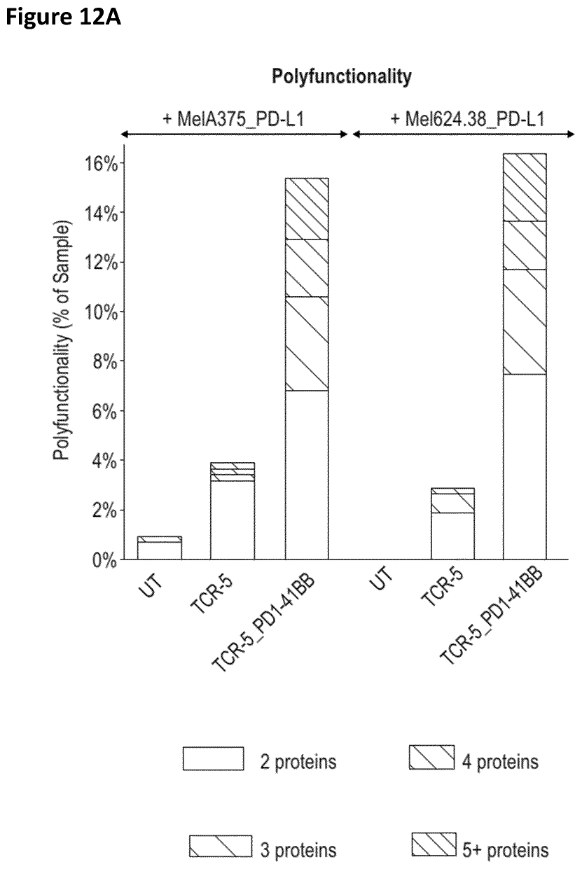

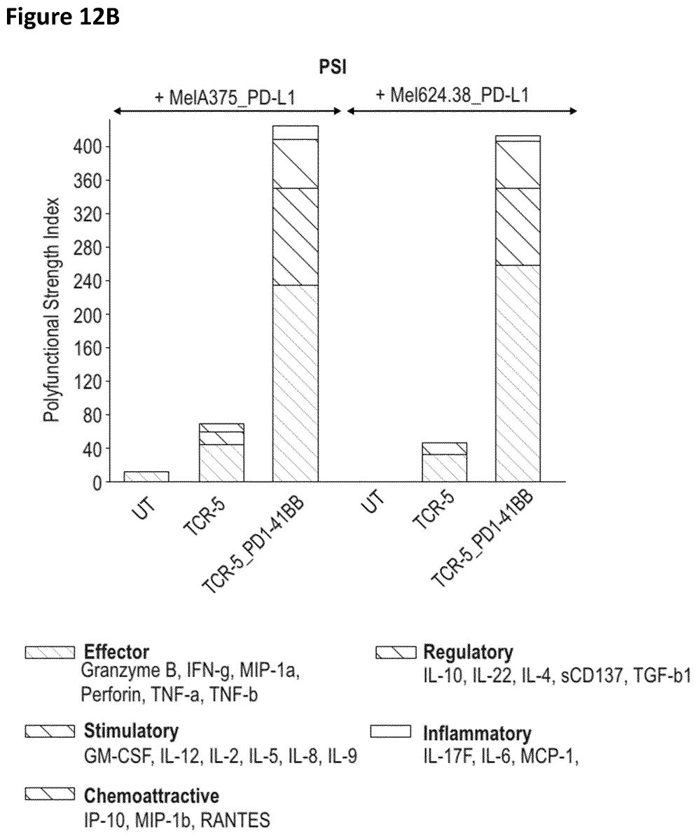

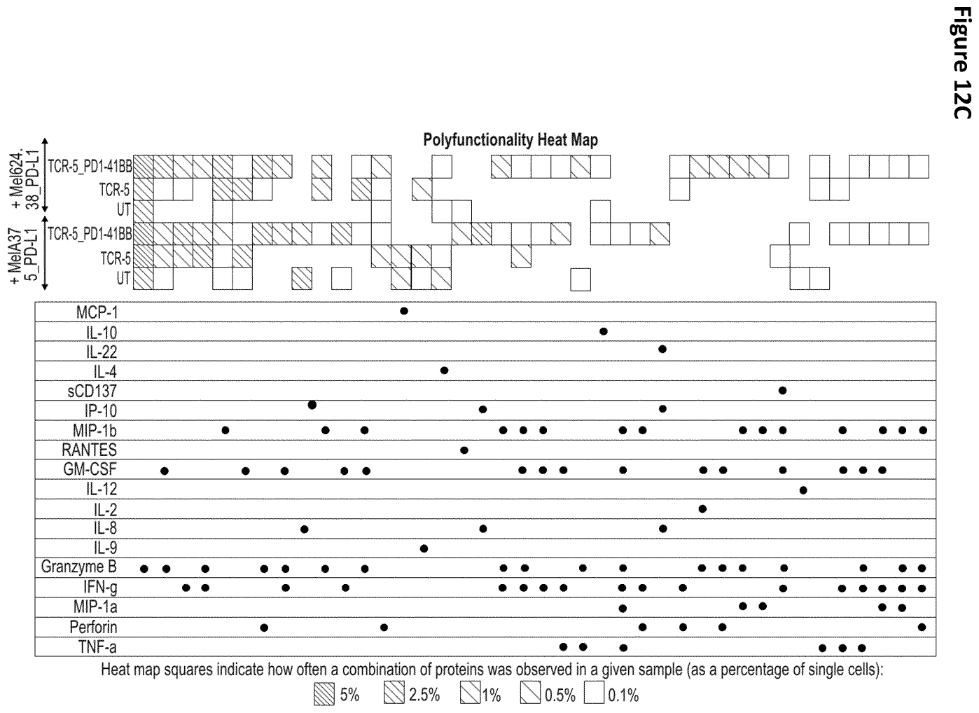

T2 PD-L1 cells were loaded with titrated amounts of SLLQHLIGL (SLL)-peptide (10⁻⁵ M to 10⁻¹⁰ M) and co-cultured with effector T cells expressing either no transgenic TCR (UT), only the transgenic TCR (TCR) or the combination of TCR and PD1-41BB (TCR_PD1-41BB) at an E: T of 1:1 (20.000 cells). IFN-γ ELISA was performed 20 h after co-culture and served to assess the reactivity of the effector T cells when challenged with different peptide concentrations presented by the T2_PD-L1 cells (FIG. 2). The half maximal IFN-γ release serves as measure for functional avidity of the TCR-transgenic effector T cells. In the left graph absolute IFN-γ levels are depicted, while the right graph shows the calculated the non-linear regression curve of relative values. As shown through the absolute IFN-γ values, the co-expression of PD1-41BB increases the total amount of IFN-γ secreted by the T cells compared to effectors expressing only the transgenic TCR. However, the non-linear regression curve demonstrates that the overall functional avidity is comparable irrespective of PD1-41BB expression. Therefore, the peptide sensitivity of TCR-transgenic T cells is not altered by the co-expression of the PD1-41BB switch receptor even in the presence of the ligand PD-L1.

### Example 3: HLA-A*02 Sub-Type Recognition is not Altered by Co-Expression of PD1-41BB.

The HLA-A2 protein can be encoded by different HLA-A*02 sub-alleles (HLA-A*02: XX) that result in slightly different amino acid sequences. A specific TCR that recognizes its cognate peptide in the context of HLA-A*02:01 does not necessarily recognize the peptide presented by another HLA-A*02 sub-allele. To define the genetic traits required for a successfully TCR-based cell therapy and potentially broaden the patient cohort, the TCR is characterized in the context of the most common HLA-A*02 sub-alleles (FIG. 3).

T cells expressing either no transgenic TCR (UT), only the transgenic TCR (TCR) or the combination of TCR and PD1-41BB (TCR_PD1-41BB) were co-cultured with lymphoblastoid cell lines (LCL; EBV-transformed B cells) carrying selected HLA-A*02 sub-alleles (HLA-A*02: XX) at an E: T ratio of 1:1 (20.000 cells/well). To allow recognition by the transgenic TCR, the LCL were loaded with 10-5 M SLL-peptide. IFN-γ concentrations were determined by ELISA 20 h after co-culture.

The TCR-transgenic effector T cells recognized the SLL-peptide presented by MHC molecules encoded by the HLA-A*02 sub-alleles A*02:02, A*02:04 and A*02:09 at similar levels compared to A*02:01. This recognition pattern was not altered by the co-expression of PD1-41BB and is in accordance with previous results. TCR-transgenic effector T cells recognize SLL-peptide in the context of 4 different HLA-A*02 sub-alleles independent of PD1-41BB co-expression.

### Example 4: Successful De-Risking of Potential Peptide Off-Target Toxicity.

Off-target toxicities can arise when TCRs recognize not only the specific peptide (e.g. SLL peptide), but also other peptides that share a high sequence homology to the original peptide. To identify likely peptide candidates that show a high sequence similarity with the specific peptide and might be recognized by the TCR, computational tools, like Expitope 2.0® [Jaravine V, Mösch A, Raffegerst S, et al. Expitope 2.0: a tool to assess immunotherapeutic antigens for their potential cross-reactivity against naturally expressed proteins in human tissues. BMC Cancer 2017; 17 (1): 892.], can be used. This tool predicts most likely mismatched (MM) peptides based on genomic, transcriptomic and proteomic data. By applying an Expitope 2.0® search 191 MM peptides could be identified that showed up to 4 amino acid differences compared to the specific SLL-peptide. In a pre-screening co-culture using PD-L1-transgenic T2 cells loaded with 10⁻⁶ M of the MM peptides or the SLL-peptide, 33 MM peptides were identified that were recognized by TCR-transduced T cells. Since exogenous loading of high peptide concentrations does not necessarily translate into physiological recognition of endogenously processed and presented peptides, the 33 MM peptides were examined for their potential to induce IFN-γ release by TCR-transgenic effector T cells when the epitopes (peptides) are translated from in vitro transcribed RNA (ivtRNA) and endogenously processed in the PRAME-negative target cell line SNB-19. IvtRNA coding for up to 5 MM peptides was electroporated into SNB-19 cells. The MM peptides that induced the highest IFN-γ release in TCR-transgenic T cells in the pre-screening co-culture (MM01, MM26, MM66), were tested individually as “midigene” constructs (˜400 bp). All other MM peptitdes were tested as minigene constructs (˜90 bp per peptide) coding for 5 MM peptides. A midigene construct coding for the SLL peptide was used as a positive control. To confirm successful ivtRNA transfection, all RNA constructs included an epitope recognized by a positive-control TCR. IFN-γ concentrations were determined 20 h after co-culture of the transfected SNB-19 cells with TCR-transgenic effector T cells. All transfected SNB-19 cells were recognized by the positive control TCR, confirming successful transfection (FIG. 4). SNB-19 cells transfected with the ivtRNA construct encoding the SLL-peptide were recognized by TCR-transgenic T cells with and without PD1-41BB, whereas none of the intracellular processed MM peptides were recognized. Therefore, all MM peptides could be de-risked and are not likely to cause off-target toxicities.

### Example 5: No Off-Target Toxicity was Identified Using a LCL Library Covering Frequent HLAS.

To obtain information about potential cross-reactivities of TCR-transgenic T cells with other HLA allotypes, a library of lymphoblastoid cell lines (LCLs) covering the most frequent HLA-A, -B and -C alleles in the Caucasian population was used as target cells. These LCL express a wide variety of endogenously expressed peptides and help to identify potential cross-reactivities due to recognition of endogenous peptides presented on the matched HLA-A2 molecule or the most frequent other HLA molecules. Detected cross-recognition of particular HLA allotypes would lead to an exclusion of patients with respective HLA alleles from clinical studies. T cells expressing either no transgenic TCR (UT), only the transgenic TCR (TCR) or the combination of TCR and PD1-41BB (TCR_PD1-41BB) were co-cultured with 36 different LCL at an E: T ratio of 1:1. IFN-γ concentrations were determined by ELISA 20 h after co-culture. TCR-transgenic T cells with and without PD1-41BB recognized an HLA-A*02:01 positive LCL loaded with SLL-peptide that served as a positive control (FIG. 5). Only the co-culture with one HLA-A*02:01 LCL without any exogenous peptide loading resulted in the release of IFN-γ. As low levels of PRAME-RNA could be detected in this cell line by quantitative real-time polymerase chain reaction (qPCR), the slight recognition was most likely on-target recognition of SLL-peptide presented by HLA-A2. None of the other LCL were recognized by the effector T cells expressing the transgenic TCR or the transgenic TCR in combination with PD1-41BB. Therefore, no off-target toxicities caused by the recognition of endogenous peptides presented on the matched HLA-A2 molecule or the most frequent other HLA molecules could be detected.

### Example 6: No Off-Target Toxicity was Identified Using a Panel of Normal Cells.

The aim of this experiment is to assess potential on-target/off-tumor and off-target toxicities that could be caused by PRAME-specific TCR-transgenic T cells with and without PD1-41BB. HLA-A*02:01-positive primary normal cells and induced pluripotent stem cell (iPS)-derived cell lines representing essential tissues or organs were tested for recognition by TCR-transduced T cells. In line with the properties of the individual targets, cells were seeded one to seven days prior to start of the co-culture at cell densities as per manufacturer's instructions and cultivated in monolayers in flat bottom wells. PRAME mRNA expression of all tested normal cells was analyzed by quantitative real-time polymerase chain reaction (qPCR) in order to distinguish on-target/off-tumor from potential off-target toxicities. 10⁻⁵ M peptide-loaded target cells served as internal positive control (SLL-peptide). IFN-γ concentrations were determined by ELISA 20 h after co-culture. All normal cells loaded with the specific SLL-peptide were recognized by TCR-transgenic T cells with and without PD1-41BB (FIG. 6). Without peptide-loading, only mature dendritic cells (mDC) resulted in IFN-γ levels above the background of untransduced cells. As mDC express PRAME, the recognition is due to on-target recognition of SLL-peptide presented by HLA-A2. None of the other target cells were recognized by the effector T cells expressing the transgenic TCR or the transgenic TCR in combination with PD1-41BB. Therefore, no off-target toxicities caused by the recognition of endogenous peptides was observed.

### Example 7: PD1-41BB Enhances the Specific Release of IFN-γ in Response to Tumor Cells Expressing PD-L1.

The interaction of PD-L1 on tumor cells with PD-1 on T cells usually leads to an inhibitory signal that reduces T cell activity. PD1-41BB should reverse this signal and result in increased T cell reactivity when the transgenic TCR binds to its cognate peptide-MHC complex. To test the impact of PD1-41BB co-expression on the reactivity of TCR-transgenic T cells, effector T cells with or without PD1-41BB were co-cultured with tumor cells expressing the ligand PD-L1. Tumor cells derived from various indications expressing the specific antigen PRAME at different levels were selected for this co-culture (FIG. 7A). PRAME-RNA expression levels in tumor cell lines were determined by real-time quantitative PCR and normalized to the housekeeping gene GUSB. While 10 tumor cell lines showed PRAME expression, no expression of PRAME mRNA could be detected in 4 tumor cells lines. However, these 4 PRAME-negative tumor cell lines express the PD1-41BB ligand PD-L1 and served as negative controls to ensure that PD1-41BB does not negatively impact the specificity of the transgenic TCR-T cells. PD-L1 expression levels were determined by antibody staining and subsequent flow cytometry analysis. To allow stable expression some tumor cell lines were transduced (TD) with PD-L1. While some tumor cell lines showed endogenous (end) PD-L1 expression, the expression of PD-L1 could be induced (ind) in other cell lines via treatment with IFN-γ. The IFN-γ levels used to induce expression were comparable to levels generated in a co-culture experiment with specific T cells recognizing their antigen. Endogenous PD-L1 expression levels in tumor cells could generally be further increased by IFN-γ treatment (end, ind).

To determine the impact of PD1-41BB co-expression on cytokine release, TCR-transgenic T cells with and without PD1-41BB were co-cultured with the selected HLA-A*02:01-positive tumor cell lines expressing different levels of PRAME and PD-L1 (FIG. 7B). Untransduced (UT) T cells were used as control. TCR-transgenic T cells and tumor cells were co-cultured at an E: T ratio of 1:1 (20.000 cells) and IFN-γ concentrations were determined by ELISA 20 h after co-culture. Co-expression of PD1-41BB enhanced the release of IFN-γ in response to PD-L1-positive tumor cells, indicating that the co-expression of PD1-41BB improves T cell reactivity in response to PD-L1-positive tumor cells. At the same time, PRAME-negative tumor cells that show PD-L1 expression are not recognized demonstrating that co-expression of PD1-41BB does not impact the specificity of the TCR-transgenic T cells. Increased cytokine release is only observed when the transgenic-TCR T cells recognize the specific PRAME antigen.

### Example 8: PD1-41BB Enhances the Specific Cytotoxic Response Against 3D Tumor Cells Spheroids.

To determine whether PD1-41BB co-expression has a beneficial effect on cytotoxicity, TCR-transgenic T cells were co-cultured with PD-L1-positive 3D tumor cell spheroids (FIG. 8). These 3-dimensional tumor cell spheroids should serve as an in vitro model for solid tumors. From the tumor cell panel introduced in Example 7, three HLA-A*02:01-positive tumor cell lines were selected that showed different levels of PRAME-expression and expressed a red-fluorescent protein (NucLight-Red). Cytotoxicity against the tumor spheroids was determined by loss of red fluorescence over 20 days using Incucyte Zoom® or S3® devices with images being recorded every 4 hours. To investigate the impact of PD1-41BB co-expression on T cell fitness and resilience, fresh tumor cell spheroids were transferred to the co-culture plates on day 3, 7, 10, 13 and 16. In this challenging environment with repeated exposure to tumor cells, expression of PD1-41BB has a beneficial effect on the effector function and fitness of T cells. In the course of multiple challenges with tumor cell spheroids, PD1-41BB-expressing effector T cells can control tumor cell growth better compared to effector T cells expressing only the transgenic TCR. Additionally, PRAME-negative PD-L1-positive tumor cells were not targeted by transgenic-TCR T cells independent of PD1-41BB expression. Therefore, the PD1-41BB co-expressing T cells remain strictly antigen-dependent while the specific cytotoxic response against PD-L1-positive 3D tumor cells spheroids is enhanced.

### Example 9: PD1-41BB Increases the Proliferation of TCR-Transgenic T Cells in Response to Tumor Cells Expressing PD-L1.

The increased effector functions and resilience of TCR-transgenic T cells co-expressing PD1-41BB was determined by enhanced cytokine release and cytotoxicity in response to PD-L1-positive tumor cells (Example 7, 8). Especially, better tumor cell control even after multiple challenges with tumor cell spheroids indicates an increase in T cell fitness in a suppressive tumor cell environment that might also be associated with better survival or proliferation of the T cells. The 4-1BB signaling domain included in the PD1-41BB switch receptor is known to provide co-stimulation that increases the proliferation rate of T cells (Choi et al., 4-1BB signaling activates glucose and fatty acidmetabolism to enhance CD8+ T cell proliferation; 2017). To investigate whether this increased T cell expansion can also be observed when PD1-41BB interacts with its ligand PD-L1, the TCR-transgenic T cells were co-cultured with PD-L1-positive tumor cells expressing different levels of PRAME antigen (FIG. 9). TCR-transgenic T cells and HLA-A*02:01-positive tumor cell lines were co-cultured at an E: T of 1:1 and untransduced T cells (UT) were used as control. To determine the X-fold expansion of T cells in the co-culture with PD-L1-positive tumor cells, the cells were harvested on day 7 and the total cell count was determined using the MACSQuant® X Analyzer. The flow cytometry-based cell counting allowed an easy differentiation between T cells and tumor cells that might still be present in the co-culture. As expected, untransduced T cells did not proliferate in response to PRAME-positive tumor cells since the specific TCR-stimulus required for expansion is absent. Transgenic TCR-T cells proliferated in response to PD-L1-positive tumor cells in a manner that seems to be dependent on the level of the specific antigen PRAME. The co-expression of PD1-41BB enhanced the proliferation and survival in response to PD-L1-positive tumor cells compared to T cells expressing only the transgenic TCR also in an antigen-level-dependent manner. Therefore, the expression of PD1-41BB in TCR-transgenic T cells improves the proliferation rate and contributes to better survival of the cells in a challenging tumor cell milieu containing the inhibitory PD-L1 receptor.

### Example 10: T Cells Co-Expressing PD1-41BB Show Strong Anti-Tumor Reactivity In Vivo.

Co-expression of PD1-41BB increased the anti-tumor effector functions of TCR-transgenic T cells in in vitro assays. To confirm this positive effect of PD1-41BB on TCR-transgenic T cells also in vivo, we developed a mouse model using immunodeficient (NOD/Shi-scid/IL-2Rγnull) mice and the PRAME/HLA-A*02:01-positive melanoma cell line MelA375. To mimic the immunosuppressive environment of solid tumors, MelA375 cells were transduced with PD-L1.

One week after subcutaneous injection of 5×10⁶ PD-L1-transgenic MelA375, the mice had developed palpable tumors. At this time point mice were distributed to three treatment groups with six mice each. Mice were injected with 10×10⁶ TCR-positive cells (16×10⁶ total cells) with (TCR_PD1-41BB) or without (TCR) PD1-41BB or an equal amount of untransduced T cells (UT). Tumor volume was measured 2-3 times a week. Mice with a tumor volume exceeding 1000 mm³ were sacrificed. Tumors in mice treated with untransduced T cells rapidly grew out and reached the maximal tumor volume within 2-4 weeks after T cell injection (FIG. 10). Effector T cells expressing only the transgenic TCR had little effect on the tumor growth. Only T cells co-expressing PD1-41BB could reject the tumors and were tumor-free 3.5 weeks after treatment. These data show that combining a PRAME-specific TCR showing potent in vitro anti-tumor reactivity with PD1-41BB results in highly efficient T cells that can eliminate aggressively growing tumor cells in an in vivo model.

### Example 11: TCR-T Cells Targeting PRAME Expressing PD1-41BB Show a Higher Polyfunctionality Compared to TCR-T Cells Targeting PRAME Lacking PD1-41BB.

TCR-transgenic T cells with or without PD1-41BB were analyzed regarding their single-cell polyfunctionality (release of 2 or more proteins) using the IsoLight technology (IsoPlexis). So-called “polyfunctional” T cells secrete multiple proteins (in particular cytokines and other effector proteins such as Gzm-B), thereby enabling a whole variety of effector functions and a highly effective immune response. After co-culture with the PD-L1-overexpressing, PRAME-positive MelA375 tumor cell line for 24 h, single CD8⁺ T cells were separated from tumor cells using CD8 Microbeads (Miltenyi Biotec) and loaded onto IsoCode chips (IsoPlexis). The secretion of 32 T cell cytokines/proteins was analyzed using the IsoLight device and the IsoSpeak software (IsoPlexis). TCR-T cells expressing PD1-41BB showed a higher percentage of poly-functional T cells, meaning T cell secreting 2 or more proteins, compared to TCR-T cells lacking PD1-41BB (FIG. 11A). The poly-functional strength index (PSI) was calculated by multiplying the intensities of the various secreted proteins/cytokines and the percentage of polyfunctional T cells.

TCR-T cells expressing PD1-41BB showed a higher PSI compared to TCR-T cells lacking PD1-41BB (FIG. 11B). Classification of the various proteins/cytokines released revealed a high contribution of effector proteins (Gzm-B, IFN-γ, Perforin, TNF-α, TNF-β) and stimulatory cytokines (GM-CSF, IL-2, IL-7, IL-8, IL-9, IL-12) to the superior PSI, followed by chemo-attractive cytokines (IP-10, MIP-1β). Regulatory (IL-10, IL-22) and inflammatory (IL-6, MCP-1) cytokines were released to a lower extent. Detailed analysis of the single-cell poly-protein/cytokine release using a polyfunctionality heat map showed that TCR-T cells with and without PD1-41BB differed in their poly-protein/cytokine signature (FIG. 11C). Notably, TCR-T cells expressing PD1-41BB contained a higher proportion of single cells secreting 4-10 proteins/cytokines simultaneously, pointing to a superior functionality compared to TCR-T cells lacking PD1-41BB. The main proteins/cytokines released by TCR-T cells expressing PD1-41BB were Gzm-B, IFN-γ, IP-10 and MIP-1β, belonging to the families of effector proteins and chemo-attractive cytokines and thus pointing to a high TCR-T cell functionality and potential anti-tumor activity, which was indeed also observed in in vivo experiments using the same tumor cell line (MelA375_PD-L1) (FIG. 10).

### Example 12: TCR-T Cells Targeting NY-ESO-1 Expressing PD1-41BB Show a Higher Polyfunctionality Compared to TCR-T Cells Targeting NY-ESO-1 Lacking PD1-41BB.

TCR-transgenic T cells with or without PD1-41BB were analyzed regarding their single-cell polyfunctionality (release of 2 or more proteins) using the IsoLight® technology (IsoPlexis). So-called “polyfunctional” T cells secrete multiple proteins (in particular cytokines and other effector proteins such as Gzm-B), thereby enabling a whole variety of effector functions and a highly effective immune response. After co-culture with the PD-L1-overexpressing, NY-ESO-1-positive MelA375 or Mel624.38 tumor cell line for 20 h, single CD8⁺ T cells were separated from tumor cells using CD8 Microbeads (Miltenyi Biotec) and loaded onto IsoCode chips (IsoPlexis). The secretion of 32 T cell cytokines/proteins was analyzed using the IsoLight device and the IsoSpeak software (IsoPlexis). TCR-T cells expressing PD1-41BB showed a higher percentage of poly-functional T cells, meaning T cell secreting 2 or more proteins, compared to TCR-T cells lacking PD1-41BB (FIG. 12A). The poly-functional strength index (PSI) was calculated by multiplying the intensities of the various secreted proteins/cytokines and the percentage of polyfunctional T cells. TCR-T cells expressing PD1-41BB showed a higher PSI compared to TCR-T cells lacking PD1-41BB (FIG. 12B).

Classification of the various proteins/cytokines released revealed a high contribution of effector proteins (Gzm-B, IFN-γ, MIP-1a, Perforin, TNF-α, TNF-β) and stimulatory cytokines (GM-CSF, IL-12, IL-2, IL-5, IL-8, IL-9) to the superior PSI, followed by chemo-attractive cytokines (IP-10, MIP-1β, RANTES). Regulatory (IL-4, IL-10, IL-22, sCD137, TGF-β1) and inflammatory (CP-1, IL-17F, IL8) cytokines were released to a lower extent. Detailed analysis of the single-cell poly-protein/cytokine release using a polyfunctionality heat map showed that TCR-T cells with and without PD1-41BB differed in their poly-protein/cytokine signature (FIG. 1C). Notably, TCR-T cells expressing PD1-41BB contained a higher proportion of single cells secreting 2-6 proteins/cytokines simultaneously, pointing to a superior functionality compared to TCR-T cells lacking PD1-41BB. The main proteins/cytokines released by TCR-T cells expressing PD1-41BB were Gzm-B, IFN-γ, GM-CSF and MIP-1β, belonging to the families of effector proteins and stimulatory cytokines and thus pointing to a high TCR-T cell functionality and potential anti-tumor activity.

The application describes the following items:

Item 1: A cell comprising
(A) a PRAME-specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
(B) a chimeric co-stimulatory receptor comprising
   an extracellular domain containing a polypeptide derived from PD-1,
   a transmembrane domain, and
   an intracellular domain containing a polypeptide derived from 4-1BB.

Item 2: Cell according to item 1, wherein the TCR is capable of binding to a PRAME peptide having the amino acid sequence SLLQHLIGL (SEQ ID NO: 1) or a portion thereof, or its HLA-A2 bound form.

Item 3: Cell according to item 2, wherein the HLA-A2 is an HLA-A*02:01, HLA-A*02:02, HLA-A*02:04 or HLA-A*02:09 encoded molecule.

Item 4: Cell according to any one of items 1 to 3, wherein binding to the sequence SLLQHLIGL (SEQ ID NO: 1) or a portion thereof, or its HLA-A2 bound form induces IFN-γ secretion by cells transduced or transfected with the TCR.

Item 5: Cell according to any one of the preceding items, wherein the TCR comprises a variable TCR α region having an amino acid sequence which is at least 80% identical to SEQ ID NO: 8 and a variable TCR β region having an amino acid sequence which is at least 80% identical to SEQ ID NO: 9.

Item 6: Cell according to any one of the preceding items, wherein the TCR comprises a variable TCR α region having the amino acid sequence of SEQ ID NO: 8 and a variable TCR β region having the amino acid sequence of SEQ ID NO: 9.

Item 7: Cell according to any one of the preceding items, wherein the TCR comprises a constant TCR α region having an amino acid sequence which is at least 80% identical to SEQ ID NO: 10 and a constant TCR β region having an amino acid sequence which is at least 80% identical to SEQ ID NO: 11.

Item 8: Cell according to any one of the preceding items, wherein the TCR comprises, a constant TCR α region having the amino acid sequence of SEQ ID NO: 10 and a constant TCR β region having the amino acid sequence of SEQ ID NO: 11.

Item 9: Cell according to any one of the preceding items, wherein the extracellular domain containing a polypeptide derived from PD-1 comprises the sequence of SEQ ID NO: 28.

Item 10: Cell according to any one of the preceding items, wherein the intracellular domain containing a polypeptide derived from 4-1BB comprises the sequence of SEQ ID NO: 32.

Item 11: Cell according to any one of the preceding items, wherein the transmembrane domain is derived from PD-1.

Item 12: Cell according to any one of the preceding items, wherein the transmembrane domain containing a polypeptide derived from PD-1 comprises the sequence of SEQ ID NO: 30.

Item 13: Cell according to any one of the preceding items, wherein the chimeric co-stimulatory receptor comprises the sequence of SEQ ID NO: 26.

Item 14: A composition comprising
a nucleic acid encoding a PRAME-specific TCR comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB.

Item 15: A nucleic acid comprising
a nucleic acid encoding a PRAME-specific TCR comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
a nucleic acid encoding a chimeric co-stimulatory receptor comprising
   an extracellular domain containing a polypeptide derived from PD-1,
   a transmembrane domain, and
   an intracellular domain containing a polypeptide derived from 4-1BB

Item 16: Composition according to item 14 or nucleic acid according to item 15, wherein the TCR is capable of binding to a PRAME peptide having the amino acid sequence SLLQHLIGL (SEQ ID NO: 1) or a portion thereof, or its HLA-A2 bound form.

Item 17: Composition or nucleic acid according to item 16, wherein the HLA-A2 is an HLA-A*02:01, HLA-A*02:02, HLA-A*02:04 or HLA-A*02:09 encoded molecule.

Item 18: Composition according to items 14 and 16 to 17 or nucleic acid according to items 15 to 17, wherein binding to sequence SLLQHLIGL (SEQ ID NO: 1) or a portion thereof, or its HLA-A2 bound form induces IFN-γ secretion by cells transduced or transfected with the TCR.

Item 19: Composition according to items 14 and 16 to 18 or nucleic acid according to items 15 to 18, wherein the TCR comprises a variable TCR α region having an amino acid sequence which is at least 80% identical to SEQ ID NO: 8 and a variable TCR β region having an amino acid sequence which is at least 80% identical to SEQ ID NO: 9.

Item 20: Composition according to items 14 and 15 to 19 or nucleic acid according to items 15 to 19, wherein the TCR comprises a variable TCR α region having the amino acid sequence of SEQ ID NO: 8 and a variable TCR β region having the amino acid sequence of SEQ ID NO: 9.

Item 21: Composition according to items 14 and 15 to 20 or nucleic acid according to items 15 to 20, wherein the TCR comprises a constant TCR α region having an amino acid sequence which is at least 80% identical to SEQ ID NO: 10 and a constant TCR β region having an amino acid sequence which is at least 80% identical to SEQ ID NO: 11.

Item 22: Composition according to items 14 and 16 to 21 or nucleic acid according to items 15 to 21, wherein the TCR comprises a constant TCR α region having the amino acid sequence of SEQ ID NO: 10 and a constant TCR β region having the amino acid sequence of SEQ ID NO: 11.

Item 23: Composition according to items 14 and 16 to 22 or nucleic acid according to items 15 to 22, wherein the extracellular domain containing a polypeptide derived from PD-1 comprises the sequence of SEQ ID NO: 28.

Item 24: Composition according to items 14 and 16 to 23 or nucleic acid according to items 15 to 23, wherein the intracellular domain containing a polypeptide derived from 4-1BB comprises the sequence of SEQ ID NO: 32.

Item 25: Composition according to items 14 and 16 to 24 or nucleic acid according to items 15 to 24, wherein the transmembrane domain is derived from PD-1.

Item 26: Composition according to items 14 and 16 to 25 or nucleic acid according to items 15 to 25, wherein the transmembrane domain containing a polypeptide derived from PD-1 comprises the sequence of SEQ ID NO: 30.

Item 27: Composition according to items 14 and 16 to 26 or nucleic acid according to items 15 to 26, wherein the chimeric co-stimulatory receptor comprises the sequence of SEQ ID NO: 26.

Item 28: A vector comprising the nucleic acid according to item 15 to 27.

Item 29: A cell comprising the composition according to items 14 and 16 to 27, the nucleic acid to item 15 to 27 or the vector according to item 28.

Item 30: Cell according to items 1 to 13 and item 29 wherein the cell is a peripheral blood lymphocyte (PBL) or a peripheral blood mononuclear cell (PBMC).

Item 31: The cell according to any one of items 1 to 13 and items 29 to 30, wherein the cell is a T cell.

Item 32: Pharmaceutical composition comprising the cell according to items 1 to 13, the cell according to items 29 to 31, the composition according to item 14 and 16 to 27, the nucleic acid according to items 15 to 27 and/or the vector according to item 28.

Item 33: Pharmaceutical composition according to item 20, wherein the pharmaceutical composition comprises at least one pharmaceutically acceptable carrier.

Item 34: The cell according to items 1 to 13, the cell according to items 29 to 31, the composition according to item 14 and 16 to 27, the nucleic acid according to items 15 to 27 and/or the vector according to item 28 for use as a medicament.

Item 35: The cell according to items 1 to 13, the cell according to items 29 to 31, the composition according to item 14 and 16 to 27, the nucleic acid according to items 15 to 27 and/or the vector according to item 28 for use in the treatment of cancer.

Item 36: The cell according to items 1 to 13, the cell according to items 29 to 31, the composition according to item 14 and 16 to 27, the nucleic acid according to items 15 to 27 and/or the vector according to item 28 for use in the treatment of cancer, wherein the cancer is preferably selected from the group consisting of melanoma, bladder carcinoma, colon carcinoma, and breast adenocarcinoma, sarcoma, prostate cancer, uterine cancer, uveal cancer, uveal melanoma, squamous head and neck cancer, synovial carcinoma, Ewing's sarcoma, triple negative breast cancer, thyroid cancer, testicular cancer, renal cancer, pancreatic cancer, ovarian cancer, esophageal cancer, non-small-cell lung cancer, non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia, preferably wherein the cancer is selected from the group consisting of NSCLC, SCLC, breast, ovarian or colorectal cancer, sarcoma or osteosarcoma.

Item 37: A cell population comprising cells expressing
(A) a PRAME specific T cell receptor (TCR) comprising
   a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
(B) a chimeric co-stimulatory receptor comprising
   an extracellular domain containing a polypeptide derived from PD-1,
   a transmembrane domain, and
   an intracellular domain containing a polypeptide derived from 4-1BB, wherein the cell population comprises cells which secrete at least two proteins.

Item 38: The cell population according to item 37, wherein the cell population comprises cells which secrete at least three proteins.

Item 39: The cell population according to item 38, wherein the cell population comprises cells which secret at least four proteins.

Item 40: The cell population according to item 39, wherein the cell population comprises cells which secret at least five proteins.

Item 41: The cell population according to item 40, wherein the proteins a selected from the group of effector proteins, stimulatory cytokines and chemo-attractive cytokines.

Item 42: The cell population according to item 41, wherein the effector proteins are selected from the group consisting of Gzm-B, IFN-γ, Perforin, TNF-α, TNF-β.

Item 43: The cell population according to item 41 or 42, wherein the stimulatory cytokines are selected from the group consisting of GM-CSF, IL-2, IL-7, IL-8, IL-9, IL-12

Item 44: The cell population according to items 41 to 43, wherein the chemo-attractive cytokines are selected from IP-10 and MIP-1β.

Item 45: The cell population according to any one of the preceding items, wherein each of the cells secreting at least two proteins, secretes at least one of the proteins selected from the group consisting of IFN-γ, Gzm-B, and IP-10,

Item 46: The cell population according to any one of the preceding items, wherein each of the cells secreting at least two proteins, secretes at least one of the proteins selected from the group consisting of IFN-γ and Gzm-B.

Item 47: The cell population according to anyone of the preceding items, wherein the TCR is capable of binding to a PRAME peptide having the amino acid sequence SLLQHLIGL (SEQ ID NO: 1) or a portion thereof, or its HLA-A2 bound form.

Item 48: The cell population according to anyone of the preceding items, wherein the extracellular domain containing a polypeptide derived from PD-1 comprises the sequence of SEQ ID NO: 28 and wherein the intracellular domain containing a polypeptide derived from 4-1BB comprises the sequence of SEQ ID NO: 32.

Item 49: The cell population according to anyone of the preceding items, wherein the transmembrane domain is derived from PD-1, wherein preferably the transmembrane domain containing a polypeptide derived from PD-1 comprises the sequence of SEQ ID NO: 30, preferably wherein the chimeric co-stimulatory receptor comprises the sequence of SEQ ID NO: 26.

Item 50: The cell population according to anyone of the preceding items for use in the treatment of cancer.

1. A cell population comprising cells expressing
(A) an antigen specific TCR, and
(B) a chimeric co-stimulatory receptor,
wherein the cell population comprises cells which secrete at least two proteins.

2. A cell population according to claim 2, wherein the chimeric co-stimulatory receptor comprises
an extracellular domain containing a polypeptide derived from PD-1,
a transmembrane domain, and
an intracellular domain containing a polypeptide derived from 4-1BB, wherein the cell population comprises cells which secrete at least two proteins.

3. A cell population according to claim 2, wherein the chimeric co-stimulatory receptor comprises
an extracellular domain containing a polypeptide derived from PD-1,
a transmembrane domain is derived from PD-1, and
an intracellular domain containing a polypeptide derived from 4-1BB, wherein the cell population comprises cells which secrete at least two proteins.

4. A cell population according to claim 3, comprising cells expressing
(A) a NY-ESO-1/LAGE-1 specific TCR, comprising
a TCR α chain comprising a CDR1 having the amino acid sequence of SEQ ID NO: 35, a CDR 2 having the amino acid sequence of SEQ ID NO: 36 and a CDR 3 having the sequence of SEQ ID NO: 37; and
a TCR β chain comprising a CDR1 having the amino acid sequence of SEQ ID NO: 38, a CDR 2 having the amino acid sequence of SEQ ID NO: 39 and a CDR 3 having the sequence of SEQ ID NO: 40; and
(B) a chimeric co-stimulatory receptor comprising
an extracellular domain containing a polypeptide derived from PD-1,
a transmembrane domain, and
an intracellular domain containing a polypeptide derived from 4-1BB, wherein the cell population comprises cells which secrete at least two proteins.

5. A cell population according to claims 1 to 3, comprising cells expressing
(A) a PRAME specific T cell receptor (TCR) comprising
a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
(B) a chimeric co-stimulatory receptor comprising
an extracellular domain containing a polypeptide derived from PD-1,
a transmembrane domain, and
an intracellular domain containing a polypeptide derived from 4-1BB, wherein the cell population comprises cells which secrete at least two proteins.

6. The cell population according to any one of the preceding claims, wherein the cell population comprises cells which secrete at least three proteins.

7. The cell population according to anyone of the preceding claims, wherein the cell population comprises cells which secret at least four proteins.

8. The cell population according to anyone of the preceding claims, wherein the cell population comprises cells which secret at least five proteins.

9. The cell population according to anyone of the preceding claims, wherein the proteins are selected from the group of effector proteins, stimulatory cytokines and chemo-attractive cytokines.

10. The cell population according to claim 9, wherein the effector proteins are selected from the group consisting of Gzm-B, IFN-γ, Perforin, TNF-α, TNF-β, MIP-1α.

11. The cell population according to claim 10, wherein the effector proteins are selected from the group consisting of Gzm-B, IFN-γ, Perforin, TNF-α, TNF-β.

12. The cell population according to claims 9 to 11, wherein the stimulatory cytokines are selected from the group consisting of GM-CSF, IL-2, IL-5, IL-7, IL-8, IL-9, IL-12.

13. The cell population according to claims 9 to 12, wherein the stimulatory cytokines are selected from the group consisting of GM-CSF, IL-2, IL-7, IL-8, IL-9, IL-12.

14. The cell population according to claims 9 to 13, wherein the chemo-attractive cytokines are selected from IP-10 and MIP-1B.

15. The cell population according to any one of the preceding claims, wherein the proteins are selected from the group consisting of Gzm-B, IFN-γ, Perforin, TNF-α, TNF-β, MIP-1a, GM-CSF, IL-2, IL-5, IL-7, IL-8, IL-9, IL-12, IP-10 and MIP-1B.

16. The cell population according to any one of the preceding claims, wherein the proteins are selected from the group consisting of Gzm-B, IFN-γ, Perforin, TNF-α, TNF-β GM-CSF, IL-2, IL-8, MIP-1B and IP-10.

17. The cell population according to any one of the preceding claims, wherein the proteins are selected from the group consisting of Gzm-B, IFN-γ, Perforin, TNF-α, TNF-β GM-CSF, IL-2, MIP-1B.

18. The cell population according to any one of the preceding claims, wherein the proteins are selected from the group consisting of IFN-γ, Gzm-B, and IP-10.

19. The cell population according to any one of the preceding claims, wherein the proteins are selected from the group consisting of IFN-γ and Gzm-B.

20. The cell population according to any one of claims 4 and 6 to 19, wherein the TCR is capable of binding to NY-ESO peptide having the amino acid sequence set out in SEQ ID NO: 34 or a portion thereof, preferably its HLA-A2 bound form.

21. The cell population according to claim 20, wherein the HLA-A2 is an HLA-A*02:01, HLA-A*02:02, HLA-A*02:04 or HLA-A*02:09 encoded molecule preferably HLA-A*2:01 encoded molecule.

22. The cell population according to anyone of claims 5 to 19, wherein the TCR is capable of binding to a PRAME peptide having the amino acid sequence SLLQHLIGL (SEQ ID NO: 1) or a portion thereof, preferably its HLA-A2 bound form.

23. The cell population according to claim 22, wherein the HLA-A2 is an HLA-A*02:01, HLA-A*02:02, HLA-A*02:04 or HLA-A*02:09 encoded molecule.

24. The cell population according to anyone of claims 3 to 23, wherein the extracellular domain containing a polypeptide derived from PD-1 comprises the sequence of SEQ ID NO: 28 and wherein the intracellular domain containing a polypeptide derived from 4-1BB comprises the sequence of SEQ ID NO: 32.

25. The cell population according to anyone of claims 3 to 24, wherein the transmembrane domain is derived from PD-1, wherein preferably the transmembrane domain containing a polypeptide derived from PD-1 comprises the sequence of SEQ ID NO: 30, preferably wherein the chimeric co-stimulatory receptor comprises the sequence of SEQ ID NO: 26.

26. The cell population according to anyone of the preceding claims for use in the treatment of cancer.

27. A target specific immune cell expressing
(A) an antigen specific TCR, and
(B) a chimeric co-stimulatory receptor,
wherein the target specific immune cell secretes at least two proteins.

28. The target specific immune cell according to claim 27, wherein the chimeric co-stimulatory receptor comprises
an extracellular domain containing a polypeptide derived from PD-1,
a transmembrane domain, and
an intracellular domain containing a polypeptide derived from 4-1BB, wherein the target specific immune cell secretes at least two proteins.

29. The target specific immune cell according to claim 28, wherein the chimeric co-stimulatory receptor comprises
an extracellular domain containing a polypeptide derived from PD-1,
a transmembrane domain is derived from PD-1, and
an intracellular domain containing a polypeptide derived from 4-1BB, wherein the target specific immune cell secretes at least two proteins.

30. The target specific immune cell according to any one of claims 27 to 29, wherein the target specific immune cell expresses
(A) a NY-ES O-1/LAGE-1 specific TC, comprising
a TCR α chain comprising a CDR1 having the amino acid sequence of SEQ ID NO: 35, a CDR 2 having the amino acid sequence of SEQ ID NO: 36 and a CDR 3 having the sequence of SEQ ID NO: 37; and
a TCR β chain comprising a CDR1 having the amino acid sequence of SEQ ID NO: 38, a CDR 2 having the amino acid sequence of SEQ ID NO: 39 and a CDR 3 having the sequence of SEQ ID NO: 40;
(B) a chimeric co-stimulatory receptor comprising
an extracellular domain containing a polypeptide derived from PD-1,
a transmembrane domain, and
an intracellular domain containing a polypeptide derived from 4-1BB, wherein the target specific immune cell secretes at least two proteins.

31. The target specific immune cell according to any one of claims 27 to 29, wherein the target specific immune cell expresses
(A) a PRAME specific T cell receptor (TCR) comprising
a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; and
(B) a chimeric co-stimulatory receptor comprising
an extracellular domain containing a polypeptide derived from PD-1,
a transmembrane domain, and
an intracellular domain containing a polypeptide derived from 4-1BB, wherein the target specific immune cell secretes at least two proteins.

32. The target specific immune cell according to any one of claims 27 to 31, wherein the target specific immune cell secretes at least three proteins.

33. The target specific immune cell according to anyone of claims 27 to 32, wherein the target specific immune cell secretes at least four proteins.

34. The target specific immune cell according to anyone of claims 27 to 33, wherein the target specific immune cell secretes at least five proteins.

35. The target specific immune cell according to anyone of claims 27 to 34, wherein the proteins are selected from the group of effector proteins, stimulatory cytokines and chemo-attractive cytokines.

36. The target specific immune cell according to claim 35, wherein the effector proteins are selected from the group consisting of Gzm-B, IFN-γ, Perforin, TNF-α, TNF-β, MIP-1α.

37. The target specific immune cell according to claim 36, wherein the effector proteins are selected from the group consisting of Gzm-B, IFN-γ, Perforin, TNF-α, TNF-β.

38. The target specific immune cell according to anyone of claims 35 to 37, wherein the stimulatory cytokines are selected from the group consisting of GM-CSF, IL-2, IL-5, IL-7, IL-8, IL-9, IL-12.

39. The target specific immune cell according to anyone of claims 35 to 38, wherein the stimulatory cytokines are selected from the group consisting of GM-CSF, IL-2, IL-7, IL-8, IL-9, IL-12.

40. The target specific immune cell according to anyone of claims 35 to 39, wherein the chemo-attractive cytokines are selected from IP-10 and MIP-1B.

41. The target specific immune cell according to any one of claims 27 to 40, wherein the proteins are selected from the group consisting of Gzm-B, IFN-γ, Perforin, TNF-α, TNF-β, MIP-1α, GM-CSF, IL-2, IL-5, IL-7, IL-8, IL-9, IL-12, IP-10 and MIP-1β.

42. The target specific immune cell according to according to anyone of claims 27 to 41, wherein the proteins are selected from the group consisting of Gzm-B, IFN-γ, Perforin, TNF-α, TNF-β GM-CSF, IL-2, IL-8, MIP-1β and IP-10.

43. The cell population according to any one of claims 27 to 42, wherein the proteins are selected from the group consisting of Gzm-B, IFN-γ, Perforin, TNF-α, TNF-β GM-CSF, IL-2, MIP-1β.

44. The target specific immune cell according to any one of claims 27 to 43, wherein the proteins are selected from the group consisting of IFN-γ, Gzm-B, and IP-10.

45. The target specific immune cell according to any one of claims 27 to 44, wherein the proteins are selected from the group consisting of IFN-γ and Gzm-B.

46. The target specific immune cell according to any one of claims 30 and 32 to 45, wherein the TCR is capable of binding to NY-ESO peptide having the amino acid sequence set out in SEQ ID NO: 34 or a portion thereof, preferably its HLA-A2 bound form.

47. The target specific immune cell according to claim 46, wherein the HLA-A2 is an HLA-A*02:01, HLA-A*02:02, HLA-A*02:04 or HLA-A*02:09 encoded molecule preferably HLA-A*2:01 encoded molecule.

48. The target specific immune cell according to anyone of claims 31 to 45, wherein the TCR is capable of binding to a PRAME peptide having the amino acid sequence SLLQHLIGL (SEQ ID NO: 1) or a portion thereof, preferably its HLA-A2 bound form.

49. The target specific immune cell according to claim 48, wherein the HLA-A2 is an HLA-A*02:01, HLA-A*02:02, HLA-A*02:04 or HLA-A*02:09 encoded molecule.

50. The target specific immune cell according to anyone of claims 29 to 49, wherein the extracellular domain containing a polypeptide derived from PD-1 comprises the sequence of SEQ ID NO: 28 and wherein the intracellular domain containing a polypeptide derived from 4-1BB comprises the sequence of SEQ ID NO: 32.

51. The target specific immune cell according to anyone of claims 29 to 50, wherein the transmembrane domain is derived from PD-1, wherein preferably the transmembrane domain containing a polypeptide derived from PD-1 comprises the sequence of SEQ ID NO: 30, preferably wherein the chimeric co-stimulatory receptor comprises the sequence of SEQ ID NO: 26.

52. The target specific immune cell according to anyone of the claims 27 to 51, wherein the target specific immune cell is a lymphocyte.

53. The target specific immune cell according to anyone of the claims 27 to 52 for use in the treatment of cancer.


SEQUENCE LISTING (45 sequences)

SEQ ID NO: 1 - protein, 9 aa, Homo sapiens
SLLQHLIGL

SEQ ID NO: 2 - protein, 5 aa, Homo sapiens
SIFNT

SEQ ID NO: 3 - protein, 7 aa, Homo sapiens
LYKAGEL

SEQ ID NO: 4 - protein, 16 aa, Homo sapiens
CAGLADYGGSQGNLIF

SEQ ID NO: 5 - protein, 5 aa, Homo sapiens
SGDLS

SEQ ID NO: 6 - protein, 6 aa, Homo sapiens
YYNGEE

SEQ ID NO: 7 - protein, 15 aa, Homo sapiens
CASSVWASGGYEQYF

SEQ ID NO: 8 - protein, 133 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGLADYGGSQGNLIFGKGTKLSVKPN

SEQ ID NO: 9 - protein, 133 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVWASGGYEQYFGPGTRLTVT

SEQ ID NO: 10 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 11 - protein, 179 aa, synthetic construct
EDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSRSYHQGVLSATILYEILLGKATLYAVLVSALVLMAMVKRKDSRG

SEQ ID NO: 12 - DNA, 15 bp, synthetic construct
  source 1..15: mol_type = other DNA
agcatatttaacacc

SEQ ID NO: 13 - DNA, 21 bp, synthetic construct
  source 1..21: mol_type = other DNA
ttatataaggctggtgaattg

SEQ ID NO: 14 - DNA, 48 bp, synthetic construct
  source 1..48: mol_type = other DNA
tgtgccggcctggccgattacggcggctctcagggaaatctgatcttc

SEQ ID NO: 15 - DNA, 15 bp, synthetic construct
  source 1..15: mol_type = other DNA
tctggagacctctct

SEQ ID NO: 16 - DNA, 18 bp, synthetic construct
  source 1..18: mol_type = other DNA
tattataatggagaagag

SEQ ID NO: 17 - DNA, 45 bp, synthetic construct
  source 1..45: mol_type = other DNA
tgtgcctctagcgtgtgggcctctggcggctacgagcagtatttt

SEQ ID NO: 18 - DNA, 399 bp, synthetic construct
  source 1..399: mol_type = other DNA
atgctgctggaacatctgctgatcatcctgtggatgcagctgacctgggtttccggccagcagctgaatcagagccctcagagcatgttcatccaagaaggcgaggacgtttccatgaattgcaccagcagcagcatcttcaacacctggctgtggtacaagcaggaccctggcgaaggaccagtgctgctgatcgccttgtacaaagccggcgagctgaccagcaacggcagactgacagcccagttcggcattacccggaaggacagcttcctgaacatctccgccagcattccctccgacgtgggcatctatttttgtgccggcctggccgattacggcggctctcagggaaatctgatcttcggcaagggcaccaagctgagcgtgaagcccaac

SEQ ID NO: 19 - DNA, 399 bp, synthetic construct
  source 1..399: mol_type = other DNA
atgggcttcagactgctgtgctgcgtggccttttgtctgcttggagccggacctgtggatagcggcgttacccagacacctaagcacctgatcacagccacaggccagcgcgtgaccctgagatgttctcctagaagcggcgacctgagcgtgtactggtatcagcagtctctggaccagggcctgcagttcctgatccagtactacaacggcgaggaaagagccaagggcaacatcctggaacggttcagcgcccagcagttcccagatctgcacagcgagctgaacctgagcagcctggaactgggagatagcgccctgtacttctgtgcctctagcgtgtgggcctctggcggctacgagcagtattttggccctggcaccagactgaccgtgacc

SEQ ID NO: 20 - DNA, 420 bp, synthetic construct
  source 1..420: mol_type = other DNA
attcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtggaaaagagcttcgagacagacaccaacctgaacttccagaacctgtccgtgatcggcttcagaatcctgctgctgaaggtggccggcttcaacctgctgatgacactgagactgtggtccagc

SEQ ID NO: 21 - DNA, 537 bp, synthetic construct
  source 1..537: mol_type = other DNA
gaggatctgaagaacgtgttcccacctgaggtggccgtgttcgagccttctaaggccgagattgcccacacacagaaagccacactcgtgtgtctggccaccggcttctatcccgatcacgtggaactgtcttggtgggtcaacggcaaagaggtgcacagcggcgtcagcacagatccccagcctctgaaagaacagcccgctctgaacgacagccggtactgtctgagcagcagactgagagtgtccgccaccttctggcagaaccccagaaaccacttcagatgccaggtgcagttctacggcctgagcgagaacgatgagtggacccaggacagagctaagcccgtgacacagatcgtgtctgccgaagcttggggcagagccgattgtggcatcaccagcagatcttaccaccagggcgtgctgagcgccaccatcctgtatgagatcctgctgggcaaagccactctgtacgccgtgctggtgtctgccctggtgctgatggccatggtcaagcggaaggatagcagaggc

SEQ ID NO: 22 - protein, 312 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVWASGGYEQYFGPGTRLTVTEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSRSYHQGVLSATILYEILLGKATLYAVLVSALVLMAMVKRKDSRG

SEQ ID NO: 23 - DNA, 936 bp, synthetic construct
  source 1..936: mol_type = other DNA
atgggcttcagactgctgtgctgcgtggccttttgtctgcttggagccggacctgtggatagcggcgttacccagacacctaagcacctgatcacagccacaggccagcgcgtgaccctgagatgttctcctagaagcggcgacctgagcgtgtactggtatcagcagtctctggaccagggcctgcagttcctgatccagtactacaacggcgaggaaagagccaagggcaacatcctggaacggttcagcgcccagcagttcccagatctgcacagcgagctgaacctgagcagcctggaactgggagatagcgccctgtacttctgtgcctctagcgtgtgggcctctggcggctacgagcagtattttggccctggcaccagactgaccgtgaccgaggatctgaagaacgtgttcccacctgaggtggccgtgttcgagccttctaaggccgagattgcccacacacagaaagccacactcgtgtgtctggccaccggcttctatcccgatcacgtggaactgtcttggtgggtcaacggcaaagaggtgcacagcggcgtcagcacagatccccagcctctgaaagaacagcccgctctgaacgacagccggtactgtctgagcagcagactgagagtgtccgccaccttctggcagaaccccagaaaccacttcagatgccaggtgcagttctacggcctgagcgagaacgatgagtggacccaggacagagctaagcccgtgacacagatcgtgtctgccgaagcttggggcagagccgattgtggcatcaccagcagatcttaccaccagggcgtgctgagcgccaccatcctgtatgagatcctgctgggcaaagccactctgtacgccgtgctggtgtctgccctggtgctgatggccatggtcaagcggaaggatagcagaggc

SEQ ID NO: 24 - protein, 273 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGLADYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 25 - DNA, 819 bp, synthetic construct
  source 1..819: mol_type = other DNA
atgctgctggaacatctgctgatcatcctgtggatgcagctgacctgggtttccggccagcagctgaatcagagccctcagagcatgttcatccaagaaggcgaggacgtttccatgaattgcaccagcagcagcatcttcaacacctggctgtggtacaagcaggaccctggcgaaggaccagtgctgctgatcgccttgtacaaagccggcgagctgaccagcaacggcagactgacagcccagttcggcattacccggaaggacagcttcctgaacatctccgccagcattccctccgacgtgggcatctatttttgtgccggcctggccgattacggcggctctcagggaaatctgatcttcggcaagggcaccaagctgagcgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaacagcgccgtggcctggtccaacaagagcgatttcgcctgcgccaacgccttcaacaacagcattatccccgaggacacattcttccccagctccgatgtgccctgcgacgtgaagctggtggaaaagagcttcgagacagacaccaacctgaacttccagaacctgtccgtgatcggcttcagaatcctgctgctgaaggtggccggcttcaacctgctgatgacactgagactgtggtccagc

SEQ ID NO: 26 - protein, 233 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIKRGRKKLLYIFKQPFMRPVQTTQEEDGCSCRFPEEEEGGCEL

SEQ ID NO: 27 - DNA, 699 bp, synthetic construct
  source 1..699: mol_type = other DNA
atgcaaattcctcaagctccttggcctgtcgtgtgggccgttctgcaacttggatggcggcctggctggttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtcgtgggagttgttggcggactgctgggatctctggtgctgcttgtttgggtgctcgccgtgatcaagcggggcagaaagaagctgctgtacatcttcaagcagcccttcatgcggcccgtgcagaccacacaagaggaagatggctgctcctgcagattccccgaggaagaagaaggcggctgcgaactc

SEQ ID NO: 28 - protein, 170 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 29 - DNA, 510 bp, synthetic construct
  source 1..510: mol_type = other DNA
atgcaaattcctcaagctccttggcctgtcgtgtgggccgttctgcaacttggatggcggcctggctggttcctggactctcctgacagaccctggaatcctccaacattcagccccgctctgctggtggttaccgagggcgataatgccaccttcacctgtagcttcagcaacaccagcgagagcttcgtgctgaactggtacagaatgagccccagcaaccagaccgacaagctggccgcctttcctgaggatagatctcagcccggccaggactgccggttcagagttacacagctgcccaacggccgggacttccacatgtctgtcgtccgggccagaagaaacgacagcggcacatatctgtgcggcgccatttctctggcccctaaggctcagatcaaagagagcctgagagccgagctgagagtgacagaaagacgggccgaagtgcccacagctcacccttcaccttctccaagacctgccggccagttccagacactggtc

SEQ ID NO: 30 - protein, 21 aa, Homo sapiens
VGVVGGLLGSLVLLVWVLAVI

SEQ ID NO: 31 - DNA, 63 bp, synthetic construct
  source 1..63: mol_type = other DNA
gtgggagttgttggcggactgctgggatctctggtgctgcttgtttgggtgctcgccgtgatc

SEQ ID NO: 32 - protein, 42 aa, Homo sapiens
KRGRKKLLYIFKQPFMRPVQTTQEEDGCSCRFPEEEEGGCEL

SEQ ID NO: 33 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgaactc

SEQ ID NO: 34 - protein, 9 aa, Homo sapiens
SLLMWITQC

SEQ ID NO: 35 - protein, 6 aa, Homo sapiens
DRGSQS

SEQ ID NO: 36 - protein, 6 aa, Homo sapiens
IYSNGD

SEQ ID NO: 37 - protein, 9 aa, Homo sapiens
CAADDKIIF

SEQ ID NO: 38 - protein, 5 aa, Homo sapiens
SGHDY

SEQ ID NO: 39 - protein, 6 aa, Homo sapiens
FNNNVP

SEQ ID NO: 40 - protein, 10 aa, Homo sapiens
CASSRGGSGY

SEQ ID NO: 41 - protein, 129 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAADDKIIFGKGTRLHILPN

SEQ ID NO: 42 - protein, 132 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVE

SEQ ID NO: 43 - protein, 269 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAADDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 44 - protein, 308 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGGSGYTFGSGTRLTVVEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 45 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR